(19) United States
(12) Patent Application Publication
(10) Pub. No.: US 2016/0236012 A1
(43) Pub. Date: Aug. 18, 2016

(54) ULTRASOUND STIMULATION OF PANCREATIC BETA CELLS

(71) Applicant: The George Washington University a Congressionally Chartered Not-for-Profit Corporation, Washington, DC (US)

(72) Inventors: Vesna Zderic, Washington, DC (US); Ivan S. Castellanos, Washington, DC (US); Aleksandar Jeremic, Washington, DC (US)

(73) Assignee: The George Washington University a Congressionally Chartered Not-for-Profit Corporation, Washington, DC (US)

(21) Appl. No.: 15/047,511

(22) Filed: Feb. 18, 2016

Related U.S. Application Data
(60) Provisional application No. 62/117,857, filed on Feb. 18, 2015.

(51) Int. Cl. A61N 7/00; A61B 5/145

(52) U.S. Cl. CPC A61N 7/00; A61B 5/14532; A61B 5/14503; A61N 2007/0004

(57) ABSTRACT

A pancreatic beta cell stimulation system for stimulating release of insulin from pancreatic beta cells can include an ultrasonic transducer configured to be acoustically coupled to a body of a user; and an ultrasound controller configured to be in communication with the ultrasonic transducer so as to provide control signals to the ultrasonic transducer during operation. The ultrasound controller can be further configured to generate the control signals based on a planned amount of stimulation of pancreatic beta cells within the body of the user such that the control signals instruct the ultrasonic transducer to transmit ultrasound waves having selected intensity and frequency calculated to cause stimulation of the pancreatic beta cells.

### CROSS REFERENCE TO RELATED APPLICATIONS

This application claims priority to U.S. Application Ser. No. 62/117,857, filed on Feb. 18, 2015, which is hereby incorporated by reference in its entirety.

### GOVERNMENT INTEREST

The invention was made in part with Government Support under NIH grant 1R03EB019065-01. The Government has certain rights in the invention.

### TECHNICAL FIELD

The present invention relates to systems, methods and apparatuses for use in stimulating release of insulin in beta cells. More particularly, the invention relates to systems, methods and apparatuses for use in ultrasound stimulation of pancreatic beta cells.

### BACKGROUND

Type 2 diabetes mellitus is a complex metabolic disease that has reached epidemic proportions in the United States and around the world. (CDC 2013; Wild et al. 2004; Zimmet et al. 2001). The prevalence of diabetes in the United States is approximately 8.3%; worldwide, there are about 150 million cases, a number expected to double in the next 20 years. Diabetes is characterized by loss of insulin secretion and destruction of insulin-producing beta-cells. Diabetes increases the risk of development of chronic complications including atherosclerotic vascular diseases (coronary artery disease, stroke and peripheral vascular disease), retinopathy, nephropathy and neuropathies. These complications result in premature death, vision impairment and blindness, end stage kidney disease and amputation, as well as engendering enormous health care costs. Type 2 diabetes results from the interplay of multiple metabolic abnormalities including insulin resistance and progressive beta cell failure ultimately resulting in insufficient insulin secretion, decreased insulin sensitivity of peripheral tissues, and insufficient insulin secretion from pancreatic beta cells to compensate for the decreased insulin sensitivity of peripheral tissues. (Ferrannini and Mari 2004; Festa et al. 2008; Kahn 2001).

Insulin, a peptide hormone, is the main glucose regulator in human body. Insulin is synthesized and stored in secretory vesicles within the pancreatic beta-cells, and is released in a calcium-dependent manner in response to changes in blood sugar levels. An accepted model of stimulus-secretion coupling of beta-cells attributes glucose-induced insulin secretion to a sequence of events involving closure of ATP-sensitive potassium channels, membrane depolarisation, influx of calcium and a rise in cytosolic free calcium concentration, and calcium-triggered exocytosis of insulin (Henquin 2009; Sakurada et al. 1993). Over time, in patients with type 2 diabetes, large population of beta-cells undergoes apoptosis or becomes “glucose-blind.” Although remaining beta-cells in diabetic patients still produce and store insulin, glucose does not mobilize intracellular calcium and subsequently does not release insulin from these dysfunctional beta-cells (Ferrannini and Mari 2004; Israili 2011). To counteract this, some pharmaceutical approaches in the treatment of type 2 diabetes utilize sulfonylureas class of drugs which can change the permeability of beta-cell membranes (by targeting ATP sensitive potassium channels) to allow calcium influx and triggering of insulin release (Neumiller and Setter 2009). However, this class of drugs is also shown to promote failure of beta-cells (Raskin 2010).

Clinical trials have shown that intensive blood glucose control together with reduction of the other cardiovascular risk factors (hypertension, hyperlipidemia, smoking, etc.) can reduce the development of chronic complications associated with type 2 diabetes. Many classes of pharmacologic agents are now employed to control hyperglycemia in patients with type 2 diabetes including insulin sensitizers, insulin secretegogues and gastrointestinal hormone analogues and modulators. However, controlling type 2 diabetes is often difficult as pharmacological management routinely requires complex therapy with multiple medications, and loses its effectiveness over time. Further, pharmacological management may be associated with increased risks of hypoglycemia (insulin, insulin secretegogues), weight gain (insulin, thiazoli-dendiones, sufonylureas), gastrointestinal side effects (metformin, GLP-1 analogues) and other risks.

Many patients are poorly compliant with lifestyle change recommendations, and pharmacological management routinely requires complex therapy with multiple medications, and loses its effectiveness over time. Also, treatment with oral agents may become less effective over time as beta cell failure progresses. Therefore, many patients ultimately require insulin therapy. However, intensive therapy with insulin may require the injection of multiple doses of different insulin formulations and is associated with concerns including weight gain and risk of hypoglycemia. Therefore, there is a growing interest in finding alternative, non-invasive methods for treatment of diabetes, especially for improving beta cell function in patients with type 2 diabetes to restore normal blood glucose levels, and there is a growing interest in finding alternative methods for the treatment of diabetes.

Currently available interventions in the treatment of type 2 diabetes usually fail over time, and new modes of therapy are needed that will directly target the underlying causes of abnormal glucose metabolism, such as beta-cell dysfunction (Spellman 2007).

What is needed are systems, methods and apparatuses for alternative, non-invasive methods for treatment of diabetes, especially for improving beta cell function in patients with type 2 diabetes to restore normal blood glucose levels.

### SUMMARY OF THE INVENTION

A pancreatic beta cell stimulation system for stimulating release of insulin from pancreatic beta cells can include an ultrasonic transducer configured to be acoustically coupled to a body of a user; and an ultrasound controller configured to be in communication with the ultrasonic transducer so as to provide control signals to the ultrasonic transducer during operation. The ultrasound controller can be further configured to generate the control signals based on a planned amount of stimulation of pancreatic beta cells within the body of the user such that the control signals instruct the ultrasonic transducer to transmit ultrasound waves having selected intensity and frequency calculated to cause stimulation of the pancreatic beta cells.

A method of stimulating insulin release from pancreatic beta cells within a body of a subject can include determining intensity and frequency for exposure of pancreatic beta cells within the body of the subject based on a planned stimulation of pancreatic beta cells within the body of the user; and exposing the pancreatic beta cells within the body of the subject to ultrasound waves using the determined intensity and frequency.

Additional features, advantages, and embodiments of the invention are set forth or apparent from consideration of the following detailed description, drawings and claims. Moreover, it is to be understood that both the foregoing summary of the invention and the following detailed description are examples and intended to provide further explanation without limiting the scope of the invention as claimed.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows an ultrasound insulin system for stimulation of beta cells, according to an embodiment of the invention.

FIG. 2 shows an ultrasound experimental setup, according to an embodiment of the invention.

FIG. 3 is a schematic of an ultrasound experimental setup, according to an embodiment of the invention.

FIG. 4 is a diagram of an experimental setup, according to an embodiment of the invention.

FIG. 5 illustrates an experimental setup, according to an embodiment of the invention.

FIG. 6 illustrates cell viability results with respect to ultrasound treatment, according to an embodiment of the invention.

FIG. 7 shows quantification of insulin release beta cells at various ultrasound treatments, according to an embodiment of the invention.

FIG. 8 shows results of intracellular insulin content in cells exposed to ultrasound, according to an embodiment of the invention.

FIG. 9 shows detection of neurotransmitter release, according to an embodiment of the invention.

FIG. 10 shows insulin secretion from pancreatic beta cells in calcium-dependent manner, according to an embodiment of the invention.

FIG. 11 shows a schematic insulin release from beta cells based on atomic force microscopy imaging.

FIG. 12 shows an epithelial cell exposed to ultrasound, according to an embodiment of the invention.

FIG. 13a shows fluid microstreaming around a bubble, according to an embodiment of the invention.

FIG. 13b shows formation of a microjet during inertial cavitation, according to an embodiment of the invention.

FIG. 14 shows calcium transients in cultured rat pancreatic beta cells, according to an embodiment of the invention.

FIG. 15 is an illustration of an experimental setup for ultrasound stimulation of pancreatic beta cells, according to an embodiment of the invention.

FIG. 16 shows passive cavitation detection, according to an embodiment of the invention.

FIG. 17 shows an illustration of an experimental setup for ultrasound stimulation of pancreatic beta cells, according to an embodiment of the invention.

FIG. 18 shows a 3D printed exposure chamber, according to an embodiment of the invention.

FIG. 19 shows an experimental setup, according to an embodiment of the invention.

FIG. 20 shows a schematic of the setup used for experimental pressure measurements, according to an embodiment of the invention.

FIG. 21 shows results of measured vs. simulated pressures of the experimental setup, according to an embodiment of the invention.

FIG. 22 shows spectra obtained from ultrasound for passive cavitation detection, according to an embodiment of the invention.

FIG. 23 shows temperature measurements during ultrasound treatment, according to an embodiment of the invention.

FIG. 24 shows modeling of acoustic pressure maps, according to an embodiment of the invention.

FIG. 25 shows detection of cavitation cavity, according to an embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology and examples selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS® NT/98/2000/XP/Vista/Windows 7/8/etc. available from MICROSOFT® Corporation of Redmond, Wash., U.S.A. or an Apple computer executing MAC® OS from Apple® of Cupertino, Calif., U.S.A. However, the invention is not limited to be on these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but is not limited to) a hard disk drive and/or a removable storage drive, representing a floppy diskette drive, a magnetic tape drive, an optical disk drive, a compact disk drive CD-ROM, flash memory, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In alternative illustrative embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

The computer may also include an input device may include any mechanism or combination of mechanisms that may permit information to be input into the computer system from, e.g., a user. The input device may include logic configured to receive information for the computer system from, e.g. a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or other camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with a data processor either wired or wirelessly. A communications interface may allow software and data to be transferred between the computer system and external devices.

The term “data processor” is intended to have a broad meaning that includes one or more processors, such as, e.g., but not limited to, that are connected to a communication infrastructure (e.g., but not limited to, a communications bus, cross-over bar, interconnect, or network, etc.). The term data processor may include any type of processor, microprocessor and/or processing logic that may interpret and execute instructions (e.g., for example, a field programmable gate array (FPGA)). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CATS, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

The term “mobile device” is intended to be used broadly to refer to a handheld device having at least one data processor and that is configured to connect to the Internet. As used herein, mobile devices can be small enough to be handheld (such as a handheld computer) having a display screen with touch input and/or a miniature keyboard. The mobile device can be configured to run applications (“apps”) that run on an advanced mobile operating system. The term mobile device can thus include smartphones, tablets, PDAs, laptops, or other computing devices.

An objective of some embodiments is to explore a novel, non-pharmacological approach that utilizes the application of ultrasound energy to augment insulin release from pancreatic beta cells. In some embodiments, novel, non-thermal and non-invasive approach can utilizes the application of ultrasound energy to augment insulin release from beta-cells as alternative to traditional (pharmacological) approaches. Therapeutic ultrasound has been used for non-invasive and selective targeting of various internal organs including human pancreas in treatment of malignancies (Leslie and Kennedy 2007; Zhao et al. 2010). With appropriate reduction in ultrasound intensities a similar method may be adopted for stimulation of insulin release.

The mechanical effects of ultrasound have been shown to cause modification of cell membrane permeability leading to different rates of transports of ions and molecules across the membrane (Dinno et al. 1989; Hassan et al. 2010; Hsu and Huang 2004; Robinson et al. 1996; Tsukamoto et al. 2011). For example, studies have indicated that ultrasound application can lead to reversible modulation of neural tissues by activating voltage-gated sodium channels, as well as voltage-gated calcium channels (Tyler et al. 2008). These effects were followed by SNARE (Soluble NSF Attachment Protein Receptor)-mediated synaptic vesicle exocytosis indicating that ultrasound may be capable of stimulating exocytosis in other cell types such as pancreatic beta-cells (Wheeler et al. 2015). In an earlier study, ultrasound was applied to bovine adrenal chromaffin cells leading to transient influx of calcium which triggered exocytosis of catecholamines, a process known to be similar to the mechanism leading to insulin exocytosis from pancreatic beta-cells (Robinson et al. 1996). Further, it has been reported that ultrasound can be used to increase release of a protein hormone adiponectin (by approximately 70%) from adipose cells (Fujii et al. 2006).

Low-intensity therapeutic ultrasound has been utilized before in production of reversible changes in cell membrane permeability, modulation of neural tissues, and enhancement of release of the adiponectin hormone from adipose cells, and epinephrine and norepinephrine from adrenal cells. Our experiments focus on determination of effectiveness and safety of ultrasound application in stimulation of insulin release from the pancreatic beta cells.

In some embodiments, a focus on basic studies can explore whether ultrasound can lead to short-term and long-term changes in insulin secretion in a safe manner. Thus, ultrasound could be a novel and alternative method to current approaches aimed to correct secretory defects in patients with type 2 diabetes.

The ability to store insulin and release it in a regulated manner in response to changes in blood sugar levels is a major function of beta cells in the pancreas, which are the only insulin-producing cells in the human body. Patients with type 2 diabetes beta cells have a decreased ability to release insulin. Embodiments of the invention include application of ultrasound in stimulation of insulin secretion from human pancreatic beta cells as a potential novel non-pharmacological treatment for diabetes.

Energy-based methods have not been explored before for modification of insulin secretion. Further, application of therapeutic ultrasound in modification of cell secretion is a novel area of research which can potentially lead to new treatments of various endocrine and metabolic disorders. Embodiments of the invention can lead to a whole new area of therapeutic ultra-sound research. For example, low-intensity therapeutic ultrasound can be tested for enhancement of secretion of other hormones such as thyroid hormones.

In general, therapeutic ultrasound can be divided into two main categories: low-intensity ultrasound (with intensities of 0.1 to 2 W/cm², and frequencies of 1-3 MHz) used for induction of non-destructive cell and tissue effects, and high-intensity ultrasound (with intensities larger than 5 W/cm² and as high as 5,000 W/cm² and frequencies of 1-5 MHz) which is used for tissue destruction (e.g., killing of tumor cells). Low-intensity therapeutic ultrasound has been studied for delivery of thrombolytic agents, drug delivery through various biological membranes such as eye membranes, skin, and blood-brain barrier, as well as gene delivery into a variety of cells such as myocardial cells and blood vessel endothelial cells (Tachibana and Tachibana 2001, Zderic et al. 2004a, Nabili et al. 2013). Most of this work has been done in the area of wound healing indicating that changes in the cell function, induced by low-intensity therapeutic ultrasound, are mostly non-thermal in nature and due to mechanical ultrasound effects.

The mechanical effects of ultrasound have been shown to cause modification of cell membrane permeability leading to different rates of transports of ions and molecules across the membrane (Dinno et al. 1989, Hsu and Huang 2004, Robinson et al. 1996, Young and Dyson 1990). For example, a previous study indicated that ultrasound application can lead to reversible modulation of neural tissues by activating voltage-gated sodium channels, as well as voltage-gated calcium channels (Tyler et al. 2008). Chapman et al. (1980) showed that exposing thymocytes to ultrasound led to decrease in potassium ion influx together with increase in potassium efflux, without inducing cell lysis or gross membrane damage. An increase in the intracellular concentration of calcium ions was also shown to occur after exposure to therapeutic levels of ultrasound in embryonic chick fibroblasts (Dinno et al. 1989, Mortimer and Dyson 1988). Measurements performed up to 20 minutes after the treatment showed that the cells were able to reduce the calcium influx, indicating that the membrane did not suffer irreparable damage as a result of the ultrasound exposure (Mortimer and Dyson 1988). Further, a report by Fujii et al. indicated that ultrasound can be used to increase release of a protein hormone adiponectin (by approximately 70%) from adipose cells (Fujii et al. 2005, conference abstract). However, some ultrasound mechanisms leading to this hormone release may still be unknown.

Ultrasound has also been applied to bovine adrenal chromaffin cells leading to transient influx of calcium which triggered exocytosis of epinephrine and norepinephrine (Robinson et al. 1996). In this study, the secretory responses stimulated by ultrasound ceased within 60-180 seconds, indicating that they were not due to irreversible cell damage and cell death. Finally, therapeutic ultrasound was used in an in-vivo study to enhance extravasation and interstitial transport of fluorosphores (up to 100 nm in size) injected in the calf muscle of mice (Hancock et al. 2009). In some embodiments, in vivo imaging and histological analysis showed that ultrasound bioeffects caused structural alteration in muscle fiber bundles (i.e. enlarged gaps) that correlated with increased tissue permeability. These effects were shown to be reversible within 72 hours after exposure.

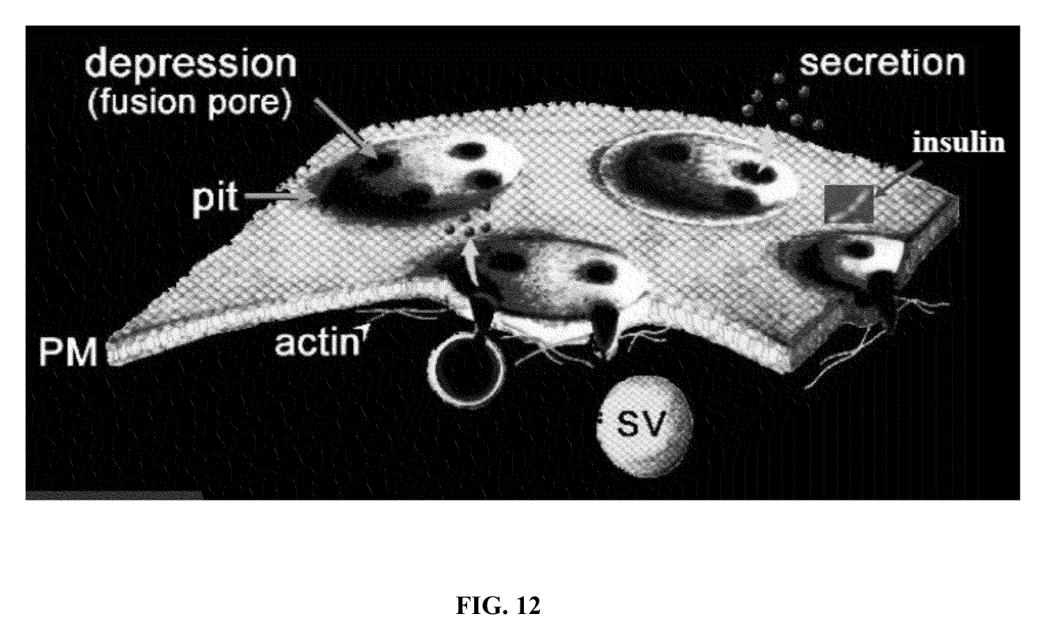

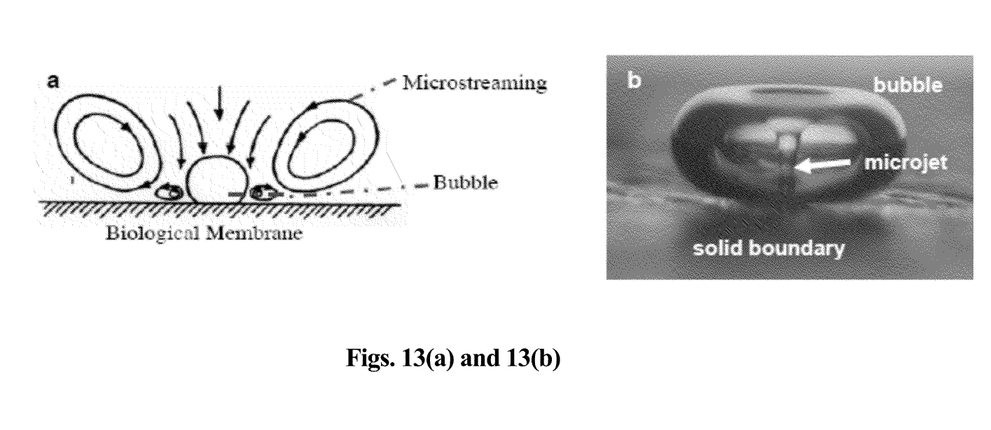

Ultrasound-induced changes in the cell membrane permeability have been strongly correlated with cavitation activity, which leads to formation of reversible pits in the cell membranes (FIG. 12) thus allowing delivery of genes, drugs, and macromolecules into the cells or release of the cell components (Guzman et al. 2002, Zderic et al. 2004a, 2004b). Specifically, a study showed that cavitation generated by ultrasound facilitates cellular incorporation of macromolecules up to 28 nm in radius through repairable micron-scale holes in the plasma membrane which were shown to reseal after 1 minute (using native cell healing response involving endogenous vesicle-based membrane resealing) (Schlicher et al. 2006). The same study further showed that cells loaded with calcein before ultrasound exposure were significantly depleted of their intracellular calcein after ultrasound exposure, thus showing that ultrasound causes bidirectional transport across plasma membrane. Cavitation-induced biological effects can be caused by microstreaming formed around a bubble oscillating in a stable manner in an ultrasound field (called stable cavitation) (FIG. 13a). Shear stresses due to microstreaming around stable cavitation can rupture cell membranes and have been shown to produce hemolysis, release of protein from bacteria, release of ATP from erythrocytes, and mechanical disruption of plant cells (Williams 1983, Miller 1987, Dalecki 2004). In addition, the bubble collapse (called inertial cavitation) may produce high pressures (108 Pa or higher), high temperatures (up to 10,000 K), and high-speed liquid jets (microjets) located in a small region (within 11.1.m3), capable of causing pits (e.g. ruptures in biological membranes) (Leighton 1994) (FIG. 13b). Studies have also shown that change in cell membrane permeability believed to be induced by acoustic cavitation and cell viability have a strong dependence on certain acoustic parameters (e.g. acoustic pressure and exposure time), thus proving useful for the design and control of ultrasound therapies (Prausnitz et al. 1998, 2001). Acoustic cavitation however, may not be the only nonthermal mechanism to increase cell and tissue permeability. Hancock et al. (2009) suggested displacements generated by acoustic radiation forces as a possible mechanism for enhanced tissue permeability. Similar ultrasound effects can enhance calcium influx and the release of insulin from pancreatic beta cells.

Dr. Vesna Zderic is an expert in various areas of therapeutic ultrasound. Specifically, she has an extensive experience in manufacturing and testing of ultrasonic transducers, conducting in-vitro, ex-vivo and in-vivo ultrasound experiments, preparation and observation of ultrasound-treated tissues and cells (using light, transmission and scanning electron microscopy), and measurement of ultrasound bioeffects (Zderic et al. 2004a, Zderic et al. 2004b, Zderic et al. 2008, Nabili et al. 2013). The co-investigator, Dr. Aleksandar Jeremic, has been successfully integrating biochemical, cell biology and high-resolution imaging approaches to study molecular mechanisms of neurotransmitter, hormone and enzyme release from various cells and tissues (Jeremic et al. 2003, Jeremic et al. 2006, Jeremic 2008, Trikha and Jeremic 2013).

In some embodiments, effectiveness and safety of ultrasound stimulation in evoking secretory responses (insulin release) in pancreatic beta cells can be assessed. Ultrasound parameters can be identified that are safe and effective at enhancing insulin secretions from suspended beta-cells, offering a potential novel method in correcting insulin deficiency in diabetics.

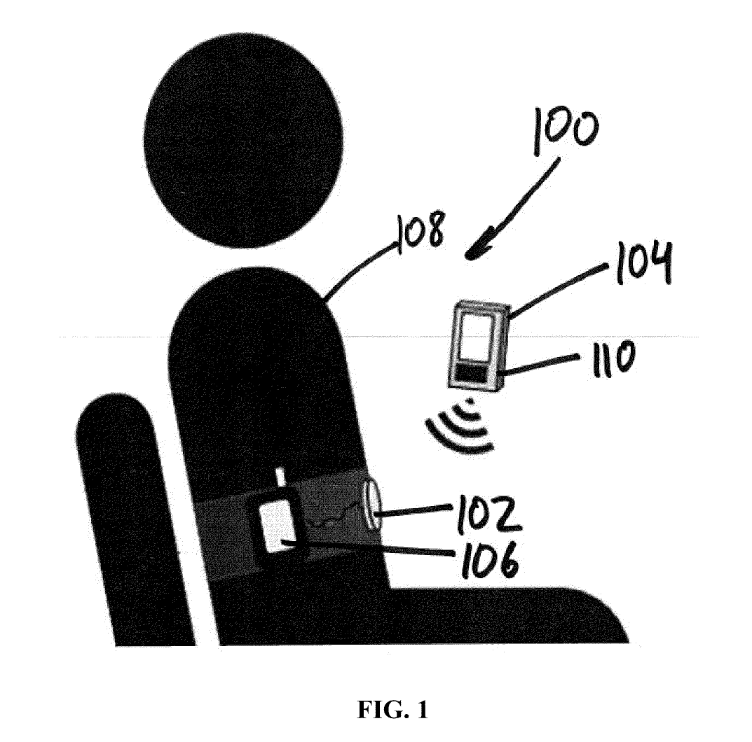

FIG. 1 shows a pancreatic beta cell stimulation system 100 for stimulating release of insulin from pancreatic beta cells. The system 100 can include an ultrasonic transducer 102 configured to be acoustically coupled to a body 108 of a user. The system 100 can include an ultrasound controller 104 configured to be in communication with the ultrasonic transducer 102. The ultrasound controller 104 can be a computer or mobile device that provides control signals to the ultrasonic transducer 102 during operation.

The ultrasound controller 104 can be further configured to generate the control signals based on a planned amount of stimulation of pancreatic beta cells within the body 108 of the user. The control signals can be analog or digital signals such that the control signals instruct the ultrasonic transducer to transmit ultrasound waves having selected intensity and frequency calculated to cause stimulation of the pancreatic beta cells. In addition to intensity and frequency, other parameters related to the ultrasound wave properties, such as amplitude and duration, can be configured to cause stimulation of the pancreatic beta cells, for example. The ultrasound waves can be generated in a continuous or pulsing mode.

Thus, minimal embodiments include an ultrasonic transducer 102 with controls optimized for stimulating insulin release by the pancreas. In such an embodiment, the ultrasonic transducer 102 could be used to treat diabetic patients periodically to increase insulin release.

In a further embodiment, the ultrasonic transducer 102 may be coupled via a controller with a glucose monitor, glucose sensing device, or glucose sensor and monitoring system 106 such that when glucose is high, the probe will be activated, thereby stimulating insulin release at appropriate times. The controller 104 may determine the appropriate parameters for the ultrasound probe activation to cause beneficial levels of insulin release. This embodiment provides for ongoing monitoring and stimulation. The ultrasonic transducer 102, controller 104, and glucose sensing device 106 can be in a wearable format enabling a patient to have in constant long-term operation.

The pancreatic beta cell stimulation system 100 can further include a data storage device 110 configured to be in communication with the ultrasound controller 104 during operation. The data storage device 110 can contain information concerning ultrasound stimulation of pancreatic beta cells. The ultrasound controller 104 can receive and use the information concerning ultrasound stimulation from the data storage device 110 to generate the control signals.

In an embodiment, the data storage device 110 can be coupled to the ultrasound controller 104. In an embodiment, the data storage device 110 can be in wireless communication with the ultrasound controller 104.

The ultrasound controller 104 can be implemented on a mobile device that is in wireless communication with the ultrasonic transducer 102.

The pancreatic beta cell stimulation system 100 can further include a glucose sensing device 106 that is configured to be in communication with the ultrasound controller 104. The glucose sensing device 106 can be attachable to the body of the user so as to sense information concerning glucose in a blood stream of the user and to provide a glucose information signal to the ultrasound controller. The ultrasound controller 104 can receive and use the glucose information signal to generate the control signals. The glucose sensing device 106 can be at least partially implantable inside the body of the user. In an embodiment, the glucose sensing device 106 can be external to the body of the user and have a needle or other sharp device to penetrate skin of the user to detect blood sugar. The glucose sensing device 106 can also include optics such as lasers that detect blood sugar by scanning the skin. In an embodiment, the ultrasound waves can be automatically generated based on the received glucose information. The waves can also be manually generated based on user input into the controller 104.

The ultrasound controller 104 can be configured to calibrate the control signals to modify the ultrasound waves based on an effect of the generated ultrasound waves on the user. The ultrasonic transducer 102 can be implanted on the surface of the pancreas and be configured to communicate with the ultrasound controller through tissue of the user. Thus, the ultrasonic transducer 102 and the glucose sensing device 106 can be at least partially implantable within the body of the user.

The ultrasound controller 104 can be further configured to generate the control signals based on a planned amount of stimulation of pancreatic beta cells within the body of the user such that the control signals instruct the ultrasonic transducer to transmit ultrasound waves for a selected duration of the stimulation of the pancreatic beta cells. For example, a user may know ahead of time how much glucose he/she may need to prepare for, and the ultrasonic transducer may be configured with selected frequency, intensity, amplitude and/or duration to provide sufficient stimulation of the pancreatic beta cells for such glucose. Thus, the ultrasonic transducer 102 can be configured to continuously generate ultrasound waves for a predetermined period of time to stimulate a predetermined amount of insulin.

The selected frequency can be in a range of about 100 kHz to 5.0 MHz, or 400 kHz to 1.0 MHz, or less than 800 kHz, or above 800 kHz. Further, the selected frequency can be about 800 kHz. The ultrasound waves can have intensities in the range of about 0.1 to 5 W/cm², about 0.1 to 2 W/cm², or above 1 W/cm² or below 1 W/cm² and/or about 1 W/cm².

The ultrasonic transducer 102 can be structured to be at least partially focusing to provide at least a degree of focus onto a sub-volume of the body of the user where pancreatic beta cells are expected to be present. The ultrasonic transducer 102 can use a direct focus on a target area of the user. The sub-volume of the body of the user can contain at least a portion of the user's pancreas. The ultrasound waves can be evenly applied to a pancreas region of the user using a uniform focus.

The sub-volume of the body of the user can contain at least a portion of implanted pancreatic beta cells. The implanted pancreatic beta cells can be derived in a number of ways, as one skilled in the art will know. For example, the beta cells can be derived from the user and cultivated outside the body. The cells can also be derived from another person, organism or be bioengineered.

The ultrasonic transducer 102 can be an array of transducer elements configured to be electronically focusable and electronically steerable. The array can include light sensing pixels at a focal plane of a lens. For example, the array can comprise a staring array, staring-plane array, focal-plane array (FPA), or focal-plane is an image sensing device.

The ultrasound sonication may be either focused or unfocused. Focused ultrasound enables targeting specifically the area of interest, in this case the pancreas.

Methods of use for the above- and hereafter-described pancreatic beta cell stimulation system are contemplated within the broad inventive principles disclosed herein. For example, a method of stimulating insulin release from pancreatic beta cells within a body of a subject can include determining amplitude and frequency for exposure of pancreatic beta cells within the body of the subject based on a planned stimulation of pancreatic beta cells within the body of the user. The method can include exposing the pancreatic beta cells within the body of the subject to ultrasound waves using the determined amplitude and frequency.

Advantages of using ultrasound to correct beta cell secretory deficiencies lie in its non-invasive and selective therapeutic targeting of human pancreas. Some embodiments of the invention comprise strategies and devices to fight diabetes. A patient-specific strategy can control the release of optimal amounts of insulin on the basis of ever-changing glucose concentrations in the blood. Ultrasound-based treatment can be used in conjunction with a minimally invasive glucose monitor and can thus non-invasively sonicate the patient's beta cells to stimulate insulin release when glucose levels in the blood are high. We will further characterize insulin release with respect to different ultrasound parameters. A defined set of parameters can optimize and control the amount of insulin release from the pancreatic beta cells while preserving cell viability. This is an important feature for clinical purposes since the optimal quantity of insulin release will depend on the concentration of glucose in the patient's blood at various times during the day. Ultrasound parameters can then be automatically modified by the device in order to supply the appropriate insulin quantity needed to reduce glucose levels in the blood as measured by the coupled glucose monitor.

Some embodiments include determining effectiveness of ultrasound stimulation of insulin release from pancreatic beta cells, for example, in human islets of Langerhans. In this embodiment, the mechanism and the extent to which ultrasound modulates excitability and insulin secretion in pancreatic beta cells can be investigated. Specifically, the ability of ultrasound to stimulate basal (constitutive) and stimulus (glucose)-evoked insulin release from suspended beta cells from human islets can be tested. The beta cell response to single and repeated ultrasound doses will be tested at various time intervals, and the long-term effects on insulin release and cell viability measured up to 3 days after ultrasound application. We will also test an effect of ultrasound on intracellular calcium mobilization in these cells as a possible mechanism for augmenting insulin secretion, as explained below. A goal here will be to establish parameters for safe stimulation of beta cell secretory activity by ultrasound.

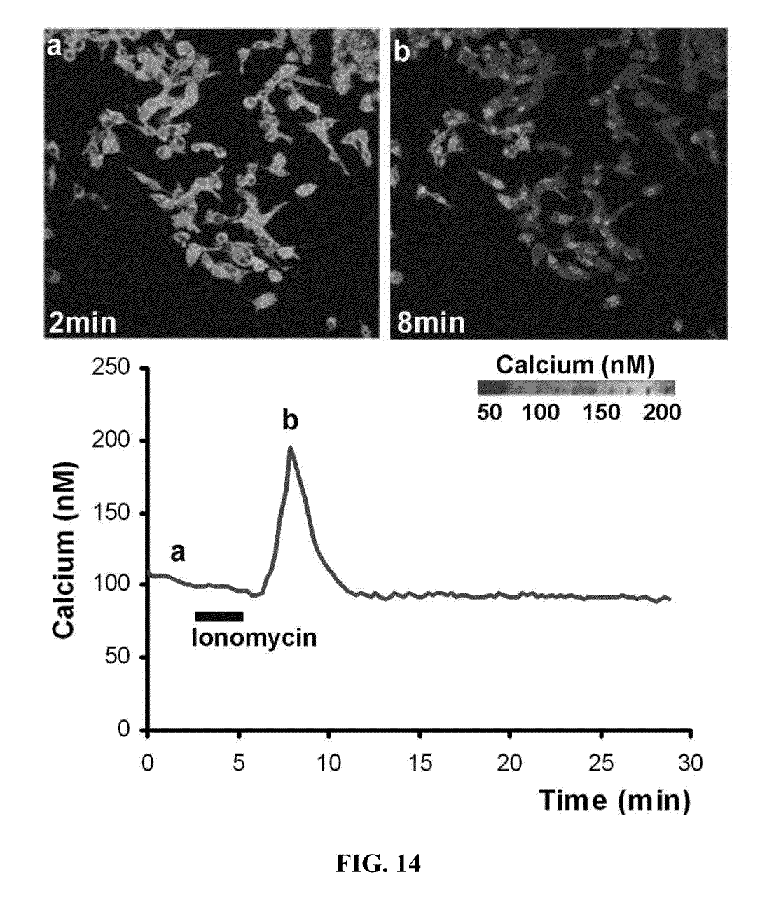

Imaging of Calcium Transients: Insulin release has been demonstrated to be calcium-dependent in human islets and cultured human cells (Henquin 2009). Because a rise in intracellular calcium is both required and self-sufficient for insulin release, approaches that modulate calcium levels in beta cells are of potential therapeutic values. Calcium levels can be modulated chemically using ionomycin (Sakurada et al. 1993), and physically using ultrasound (Robinson et al. 1996). Ionomycin is an ionophore (i.e. a channel former that produces a hydrophilic pore in the membrane, allowing calcium ions to pass through), which is used to raise intracellular levels of calcium in studies of transport through biological membranes. Ionomycin, while effective in releasing insulin, can also release other hormones and neurotransmitters (Conde et al. 2009, Sakurada et al. 1993, Yoon et al. 2008). Using ultrasound stimulation of beta cells, calcium influx can be stimulated and thus insulin release can be evoked or augmented from beta cells. Moreover, studies have shown that Ca′ influx is also crucial to initiate the cellular process to reseal the cell membrane after it has been disrupted by ultrasound-induced bioeffects (McNeil and Kirchhausen 2005, Schlicher et al. 2006). To test effect of ultrasound on beta cell excitability, calcium transients can be measured by ratiometric calcium-imaging assay as explained before (Jeremic et al. 2001). For example, we have previously used this imaging assay to observe ionomycin-promoted calcium transients in beta cells (FIG. 14). To quantify calcium changes, beta cells from the human islets will be loaded with Fura-red and Fluo-3, a cell-permeable calcium ratiometric dyes, and changes in calcium levels will be monitored using a Zeiss confocal microscopy system. Zeiss physiological software will be used to obtain images of beta cells at 480 nm and 650 nm (Paso=/F650.), and image ratio. Relative changes in fluorescence intensities (F480 nm/F650 nm) over time reflect dynamics and extent of calcium mobilization inside the cell elicited by the stimulus. Thus, imaging will be used to record and compare changes in intracellular calcium levels. In these experiments, beta cells dissociated from human islets will be suspended in a chamber and perfused with saline (modified Krebs-Ringer solution) as previously described (Jeremic et al. 2001).

Stimulatory effect of ultrasound on basal and glucose-evoked calcium mobilization can be tested alone or in combination with 5 μM ionomycin (ionomycin can be used as a positive control to trigger calcium influx). If ultrasound effect on insulin release requires calcium influx (i.e. by modulating cell excitability) then pre-incubation of cells with ionomycin should prevent or attenuate ultrasound-induced calcium transients. Removal of extracellular calcium should also abrogate stimulatory effect of ultrasound on insulin release. Toxicity assays can be performed using same cultures to determine effect of ultrasound on cell's viability as described below.

Quantification of Insulin Release: Using ELISA insulin release assay, we will determine effects of ultrasound on basal and glucose-evoked insulin release from suspended beta cells from pancreatic human islets. Briefly, beta cells will be dissociated from the islets using a cell dissociation buffer and cultured in serum-supplemented media for 24-36 h. Prior to treatments this media will be replaced with low-glucose (0.5 mM) or high-glucose (2.8 mM) KBS medium and beta cells will be exposed to ultrasound at parameters described below. Samples can be collected at 1 min intervals, and amount of insulin released in the buffer quantified by ELISA kit (Linco Research, St. Charles, Mo.). Values can be expressed as mg/ml/min of insulin released. For comparative purposes, insulin values will be also expressed as % increase from control, non-stimulated cells (assumed 100%). Ultrasound can augment both basal and glucose-evoked insulin release from beta cells, as both processes are regulated by calcium. To determine potential long-term effects of ultrasound on beta cells, secretory response of beta cells to glucose will be measured for up to 3 days following completion of ultrasound treatment.

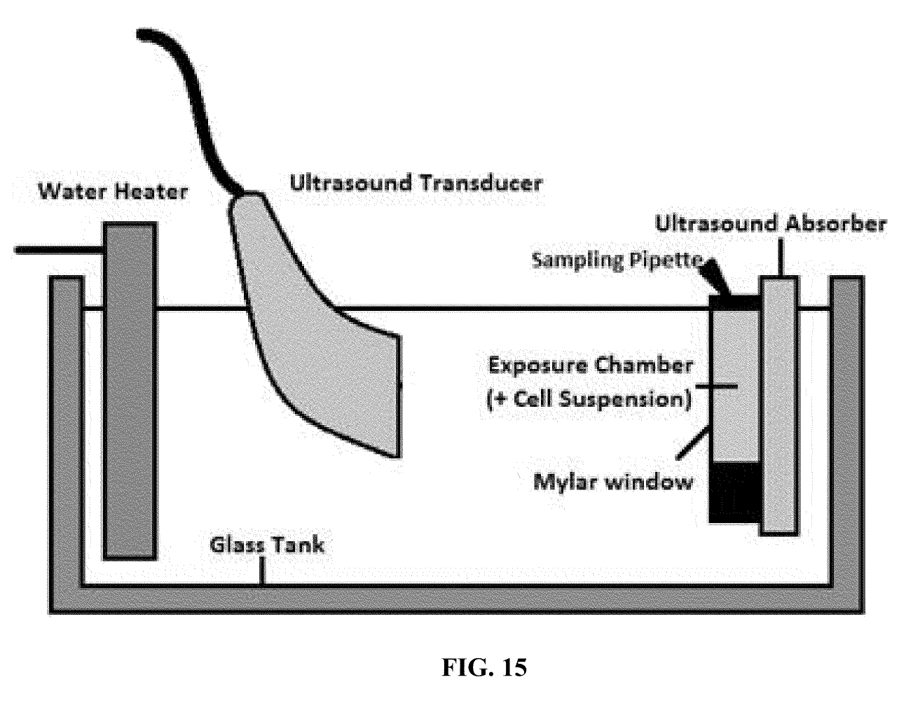

Ultrasound Application: Application of therapeutic ultrasound can lead to increase of insulin secretion from pancreatic beta cells in human islets, while maintaining cell viability. In our proposed studies, ultrasound will be applied at the range of parameters which can result in a modification of the cell response while preserving cell viability (Tachibana and Tachibana 2001; Fujii et al. 2005). Specifically, ultrasound treatment will utilize frequencies of 1 MHz or 3 MHz and intensities of 0.5 W/cm² to 2 W/cm² (continuous mode) with exposure times of 5-15 min daily, over a period of 3 consecutive days. In comparison, Fujii et al. (2005) used 1 MHz continuous wave ultrasound at intensities of 0.5 or 2.1 W/cm², in series of 3 sessions of ultrasound stimulation applied for a daily total of 15 min to promote secretion of adiponectin from adipocytes. The concentration of secreted insulin will be measured before and after ultrasound application, on each day, and for additional 3 days (once a day) following completion of ultrasound treatment. On ultrasound treatment days, the 10 pl aliquots can be withdrawn at regular (1 min) time intervals 15 min before and up to 90 min following the ultrasound stimulation, and changes in the insulin con-tent in the culturing medium will be quantified by ELISA as previously explained (Trikha and Jeremic 2013). Overall, we are planning to have 6 ultrasound treatment groups (group 1: 1 MHz, 0.5 W/cm², group 2: 3 MHz, 0.5 W/cm²; group 3: 1 MHz, 1 W/cm²; group 4: 3 MHz, 1 W/cm²; group 5: 1 MHz, 2 W/cm², group 6: 3 MHz, 2 W/cm²), and one sham treatment (control) group. The experimental setup (FIG. 15) can be similar to the setup that was used by Karshafian et al. (2009). The set-up will consist of a planar ultrasonic transducer, a glass water tank, a cell exposure chamber containing an immersible magnetic stirrer, a water heater and a thermocouple to monitor the temperature of the medium. The wall of the water tank facing the ultrasonic transducer will be covered with an ultrasound absorbing material to avoid reflection. Cells from the human islets will be placed and suspended in a cylindrical exposure chamber (12 mm inertial diameter) with acoustic transparent windows made of Mylar. The exposure chamber will be placed in the middle of the water tank. The tank will be filled with deionized water (degassed for at least 2 h) and maintained at a temperature of 37° C. (i.e. physiological temperature). The chamber containing the cell suspension will be filled with fluid and will be placed at the acoustic focus of the transducer. The cell suspension will be gently stirred with the magnetic stirrer during the experiment in order to promote uniform ultrasound exposure. We can also utilize a pulse mode of ultrasound application to allow heating dissipation if temperature increase is shown to be of concern. The ultrasonic transducer will be driven using a portable control system which can work over variety of intensities, duty cycles, and exposure times, proposed in our experiments (Sonicator 740, Mettler Electronics). The delivered ultrasound intensity will be verified using radiation force balance for acoustic power measurement, and a hydrophone for ultrasound pressure mapping.

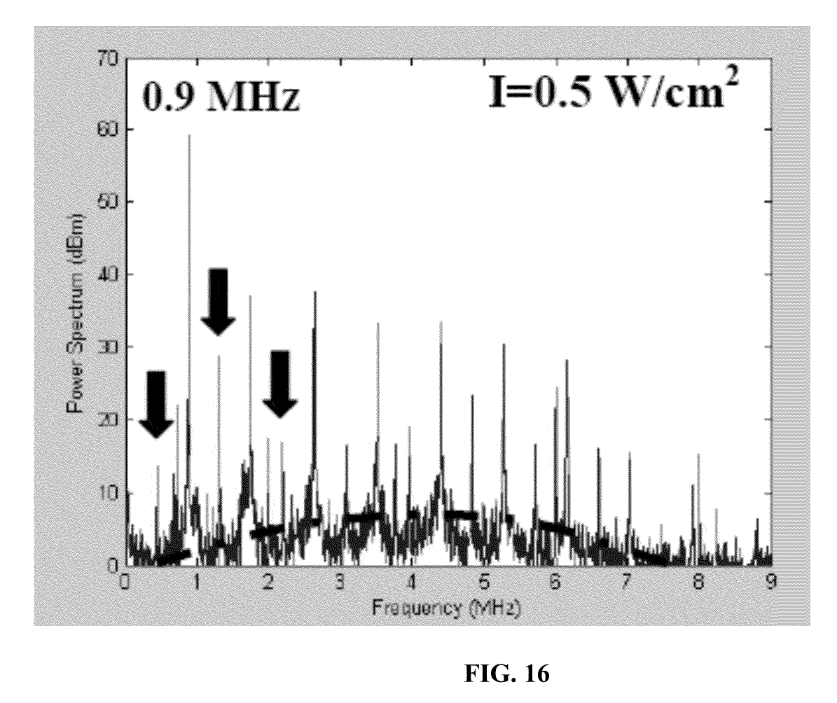

Determination of Thermal and Mechanical Ultrasound Effects: A scopemeter with thin thermocouples will be used to record temperature changes in the cell medium during sonication. In some embodiments, the temperature increase in the cell layer due to ultrasound application can be within physiological limits. The insulin release and cell viability will be correlated as a function of temperature increase. Further, in a limited set of studies the temperature of the medium will be increased using the thermal controller to mimic increases obtained during ultrasound application, to serve as a positive control. We will also apply passive cavitation detection measurements to correlate ultrasound effects on cell viability and insulin secretion with the cavitation activity. The analytical technique for quantification of cavitation activity has been described previously (Zderic et al. 2004a, 2006) (FIG. 16). Briefly, a 5-MHz hydrophone (Olympus) will be used as a cavitation detector. The hydrophone signal (obtained during ultrasound application) will be sampled, and a Fast Fourier Transform of the signal will be performed. Inertial cavitation activity will be quantified by the amount of broadband noise in a spectral band in which no harmonics or ultraharmonics (of the transducer operating frequency) are present. The power of the subharmonic (measured at the half of the transducer operating frequency) will be measured to quantify stable cavitation (Leighton 1994).

Preliminary Results in Literature: Previously published results support our hypothesis that ultrasound exposure could in fact stimulate insulin secretion from pancreatic beta cells. Ultrasound has been shown to stimulate secretion of catecholamines in calcium-dependent manner from bovine adrenal chromaffin cells (Robinson et al 1996). Their results show that ultrasound stimulation triggered secretory events in 56% of the cells when extracellular Ca2+ had entered the cell. Another study indicated that ultrasound could be used to increase the release of adiponectin from adipose cells (Fujii et al. 2005). Their study showed that adiponectin concentrations in the culture medium of the ultrasound stimulated groups increased by approximately 70%. Previous studies have also shown that ultrasound could in fact enhance release of intracellular compounds (Schlicher et al. 2006). In this study, DU 145 prostate cancer cells were loaded with calcein before being exposed to ultrasound. After sonication, these cells were significantly depleted of their intracellular calcein, thus showing that ultrasound induces bidirectional transport across the plasma membrane.

Anticipated Problems and Alternative Strategies: Preliminary data point to a physiologic, non-toxic mechanisms in regulation of cell secretion by ultrasound. However, ultrasound could modulate insulin secretion from beta cells also by affecting cell viability. To rule out this possibility, in parallel with functional studies (calcium imaging and ELISA), toxicity assays will be performed. The clear advantage of using integrated ultrasound/confocal microscope here is that optical imaging approach enables direct evaluation of efficacy of ultrasound stimulation on stimulus-secretion coupling in beta cells, and could also assist in discriminating between toxic and non-toxic cellular events. For example, calcium imaging may reveal both a physiological changes (i.e. ultrasound-evoked reversible calcium mobilization), and pathological changes in beta-cells (i.e. irreversible calcium over-load) triggered by ultrasound. Activation of voltage-dependent calcium channels by ultrasound, as shown in neurons (Tyler at al. 2008), will be explored as alternative mechanism to membrane pores for calcium influx and insulin release from beta-cells.

Furthermore, studies have demonstrated that elevated levels of intracellular Ca2+ may cause cells that would initially appear to be viable after ultrasound treatment to undergo apoptosis and die (Honda et al. 2004, Hutcheson et al. 2010). In order to account for this effect in our studies, we will also use fluorescently-conjugated annexin V as a marker of apoptosis and analyze the results via flow cytometry as previously described (Hutcheson et. al 2010).

Specific Aim 2. Determine effects of ultrasound stimulation on viability of pancreatic beta cells, for example, in human islets of Langerhans. We will test the extent to which ultrasound stimulation affects viability of beta-cells from human islets, and for example cultured beta-cells. Cell viability will be assessed for up to 3 days after ultra-sound application, using methods described below. A goal here will be to establish parameters for safe stimulation of beta cell secretory activity by ultrasound.

Cell Viability Studies: MTT cytotoxic assay will be employed to determine viability of beta cells ex-posed to different duration and frequencies of ultrasound stimulation. Caspase-3, LDH release and Annexin-apoptotic assays will be run in parallel, as an alternative to MTT assay and to investigate possible early apoptotic stages in beta-cells evoked by ultrasound stimulation. Cultured beta-cells from pancreatic human islets will be plated in modified culture flasks and maintained in serum-containing medium for 2-3 days. beta-cells will then be subjected to the ultrasound of varying intensities and different exposure times to investigate potential detrimental effect of ultrasound on cell viability.

MTT Reduction and LDH Release Cytotoxic Assays: MTT reduction cytotoxic assay will be per-formed as described before (Jeremic et al. 2001). beta-cells dissociated from human islets will be seeded at 5×104 cells/cm2 per cover slip and incubated for 24-48 h in RPMI-1640-10% FBS culture medium prior to ultrasound stimulation. Cover slips will be placed in modified culture flasks and incubated in Krebs-Ringer (insulin release) buffer. Following ultrasound stimulation MTT will be added to medium (0.5 mg/ml final) and incubation continued for additional 2 h at 37° C. Formazan crystals, a product of MTT reduction, are indicators of the pyridine nucleotide redox state of the cell. Converted crystals will be dissolved in isopropanol/HCl solution and quantified by measuring the absorbance of formazan dye at 570 nm. The extent of release or leakage of lactate dehydrogenase (LDH) from cells will be quantified using the LDH assay kit (Sigma, St. Louis, Mo.). Cells will be exposed to ultrasound as for MTT assay. Samples (control and treatments) will be collected and the LDH content determined according to manufacture instructions. The assay is based on NAD reduction to NADH and concomitant conversion of a tetrazolium dye to color product with absorbance maximum at 490 run. The amount of LDH activity in the medium is indicator of relative cell viability as well as a function of membrane integrity. Absorbance values, representing the enzymatic reduction of the MTT molecule or LDH activity in the medium by amylin, will be measured and converted into % change from control values (non-treated cells). This conversion will simplify comparisons of ultrasound effects among different cytotoxic assays.

Caspase-3/Anexin V Fluorescence Apoptotic Assay: To further investigate the effects of ultrasound on beta cell viability, Caspase-3/Anexin V Fluorescence Apoptotic assay will be performed. Thus, cultured beta cells from human islets will be exposed to ultrasound stimulation for various times and different frequencies and intensities. Following treatments, beta cells will be incubated additionally with 5 μM Annexin V rhodamine, and a 2 pM cell-permeable caspase-3 substrate for 30 min. Cells will be fixed, mounted and examined under confocal microscopy using FITC and rhodamine filter sets. The number of Caspase-3/Anexin-positive (apoptotic) cells in each well will be scored and divided by total number of cells, reflecting % of apoptotic cells in that well. Data (% of apoptotic cells) will be averaged, and expressed as mean±standard deviation of at least four independent experiments and statistically analyzed.

Atomic Force Microscopy: In a limited set of experiments, our atomic force microscope will be used to image the cell membrane surface during ultrasound treatment (at the set of ultrasound parameters found to lead to increased insulin secretion with preserved cell viability). The objective of this study is to observe the formation of ultrasound-induced pores in the cell membrane and their resealing. Previous work by Prausnitz et al. has shown (using electron and fluorescence microscopy) that these pores reseal within 1 min of ultrasound application (Schlicher et al. 2006), however the imaging modalities used in their studies were not optimal for the observation of the pores dynamics in real time. Our proposed studies with atomic force microscope would complement these previous findings by providing information about forming and closing of the pores in the cell membrane during and right after ultrasound application.

Statistical Analysis: Excel and Sigma Stat software programs will be used for plotting and analysis of data. To simplify correlations of ultrasound toxicity between different assays, data will be converted into % change from control values (non-treated cells) and values compared. Data from at least 4 separate experiments, each performed in triplicate, will be collected and arithmetical means determined. Data will be expressed as mean±standard deviation. Difference between two treatments will be established using Student's t test. For multiple comparisons between the control and treated groups data will be analyzed by one-way ANOVA followed by Dennett's post hoc test. For both tests significance will be established at p<0.05.

Ultrasound may promote secretion of insulin from human pancreatic beta cells based on previous studies which utilized adipose cells (Fujii et al. 2005). Further, cell viability may be preserved since relatively low ultrasound intensities will be used.

If proven successful our method may find a clinical application due to non-invasive nature of therapeutic ultrasound treatment of human pancreas (through an appropriate acoustic window) (Leslie and Kennedy 2007).

Ultrasound Stimulation of Pancreatic Beta Cells

A previous study has indicated that ultrasound may find an application in modification of secretion of metabolic hormones. In this study, cultures of adipose cells obtained from obese patients were exposed daily to low levels of therapeutic ultrasound. The results showed that repeated ultrasound stimulation of the adipose cells increased secretion of their hormone, adiponectin. Other studies have previously reported enhanced secretion of catecholamines from bovine adrenal chromaffin cells by ultrasound stimulation. These studies encouraged us to explore whether ultrasound may have a similar effect on insulin secretion. The main objective of this proposal is to determine the effectiveness and safety of ultrasound stimulation of insulin release from pancreatic beta-cells. By designing this novel approach we will test the ability of low-intensity therapeutic ultrasound to augment glucose-evoked insulin release from pancreatic beta cells, as a potential novel treatment for type 2 diabetes.

This approach may open new strategies to combat diabetes. Clear advantage of using ultrasound to correct beta cell secretory deficiencies lies in its non-invasive and selective therapeutic targeting of human pancreas. This approach may be used alone or in combination with existing pharmacological strategies, which makes ultra-sound attractive for targeting of type 2 diabetes in a clinically advantageous manner. Currently, focused ultrasound at high intensity levels is used for non-surgical ablation of pancreatic malignancies in patients, and with an appropriate reduction in intensity levels the same technology may eventually be used for stimulation of the beta cells in the pancreas.

Methods: Some embodiments focus on determination of effectiveness and safety of ultrasound application in stimulation of insulin release from pancreatic beta cells. ELISA insulin release assay was used to determine and quantify the effects of ultrasound on basal and glucose-evoked insulin release in cultured pancreatic beta cells. Effects of ultrasound on cell viability were assessed by employing MTT, Caspase-3, LDH release and Annexin-apoptotic cytotoxic assays. Ultrasound exposure was generated using a commercial ultrasound device (Sonicator 740, Mettler Electronics) and a planar ultrasonic transducer with center frequency of 1 MHz and intensity of 0.8 W/cm2 was used to treat the cells for 5 minutes. Insulin has been shown to be released in a calcium-dependent manner in response to changes in blood sugar levels. Therefore, our study also looked to evaluate extracellular calcium influx as a potential mechanism for enhanced ultrasound induced insulin release. Thus, calcium transients were measured and quantified by ratiometric calcium-imaging assay.

Results: Some embodiments indicate that application of therapeutic ultrasound may lead to increase of insulin secretion from beta cells in a calcium dependent manner while maintaining cell viability. ELISA results showed a 25% increase in insulin release from beta cells after ultrasound exposure for 5 minutes. Cell viability was not significantly affected during and for up to one hour after treatment. Insulin release and cell viability results will be correlated as a function of temperature increase and cavitation activity to demonstrate the potential mechanical and thermal effects of ultrasound on pancreatic beta cells.

An objective of some embodiments explored the effectiveness of a novel, non-pharmacological approach that utilizes the application of ultrasound (US) energy to augment insulin release from rat insulinoma cells (INS-1). Cells were exposed to unfocused ultrasound for 5 minutes at a peak intensity of 1 W/cm² and frequencies of 400 kHz, 600 kHz, 800 kHz and 1 MHz. Insulin release was measured with enzyme-linked immunosorbent assay (ELISA) and cell viability was assessed via trypan blue dye exclusion test. ELISA results showed marked release (>20-fold) of insulin from beta cells exposed to 400 kHz and 600 kHz ultrasound at the cost of cell viability. However, using frequencies of 1 MHz and 800 kHz resulted in approximately 1.5 and 4-fold (p<0.05) increase in insulin release respectively, as compared to controls while retaining cell viability. At these higher frequencies (≧800 kHz), insulin release was comparable to beta cells secretagogue glucose in releasing insulin from beta cells, thus operating within physiological secretory capacity of beta cells.

Methods and Materials

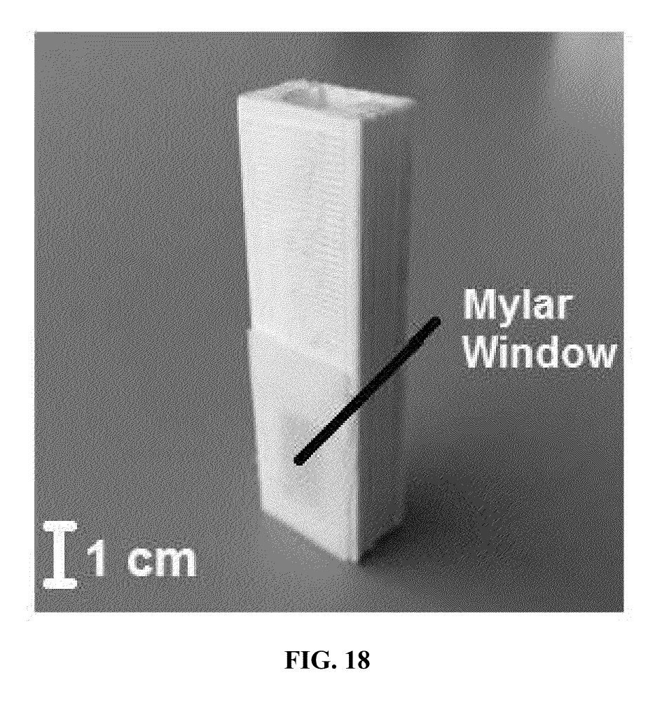

INS-1 cells, an insulin secreting insulinoma cell line, were routinely cultured in RPMI-1640 tissue culture medium (11.1 mmol glucose, pH 7.4) supplemented with 2 mM L-glutamine, 10 mM HEPES, 1 Mm sodium-pyruvate, 0.05 mM 2-mercaptoethanol and 10% fetal calf serum in a 37° C. incubator (VWR International, Radnor, Pa., USA) with 5% CO2 and 95% air. The cell lines were maintained in 14 ml of culture medium in 75 cm² sterile vented tissue culture flasks (Greiner GmbH, Pleidelsheim, Germany). Prior to treatment, trypsinated cells were collected and centrifuged for 10 min at 1000 rpm. The supernatant was removed and the cells were resuspended in 1 ml of modified Krebs bicarbonate solution (sodium chloride 138 mM, potassium chloride 5.4 mM, calcium chloride 2.6 mM, sodium bicarbonate 5 mM, magnesium chloride 1 mM, 10 mM HEPES, pH 7.4) supplemented with 0.1% bovine serum albumin. As shown in FIG. 18, the cell suspension was then loaded into a 3D printed exposure chamber made in-house out of polylactic acid (PLA) and with 0.18 mm thick acoustically transparent windows made out of Mylar® (1.5 cm×1 cm)(Karshafian et al. 2009).

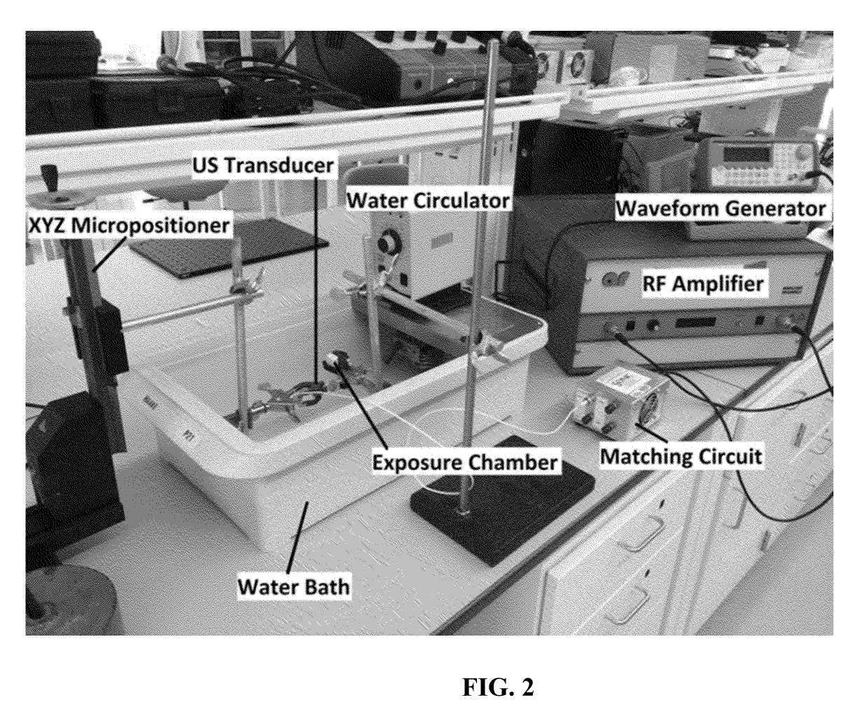

The experimental setup was placed in a water bath maintained at 37° C. (Thermo Haake DC10-P21, Fisher Scientific, Waltham, Mass., USA) as shown in FIG. 2. Circular planar ultrasonic transducers with an active diameter of 1.5 cm and center frequencies of 400 kHz, 600 kHz, 800 kHz and 1 MHz (Sonic Concepts, Inc. Bothell, Wash., USA) were directed towards the exposure chamber at a distance of 1.5 cm, 2.25 cm, 3 cm and 3.75 cm, which corresponded to their respective near-field to far-field distances (Christensen 1988). Ultrasound waveforms used as stimuli were generated using an Agilent 33220A function generator (Agilent Technologies, Santa Clara, Calif., USA) and were further amplified (50 dB gain) using a 150A100B RF amplifier (Amplifier Research, Souderton, Pa., USA). A 3-D micropositioning system with 0.025 mm resolution was used to control the distance between the ultrasonic transducer and the exposure chamber. An ultrasound absorber (Precision Acoustics LTD, Dorchester, United Kingdom) was placed in the back of the exposure chamber in order to minimize the production of standing waves. Cell samples with density roughly around 2-5×106 cells/ml were suspended in 1 ml of glucose-free Krebs bicarbonate solution (KBS), placed inside the exposure chamber and treated for 5 min with ultrasound at peak intensity of 1 W/cm² using the previously mentioned center frequencies. Aliquots of 100 μL were acquired prior to the start of the treatment (t=0 min), immediately after treatment (t=5 min) and 30 minutes after treatment (t=30 min) for analysis. To serve as positive controls, insulin release was measured from cells suspended in glucose-supplemented (12 mM) modified Krebs Ringer bicarbonate buffer (pH 7.4) (Hamid, et al. 2002).

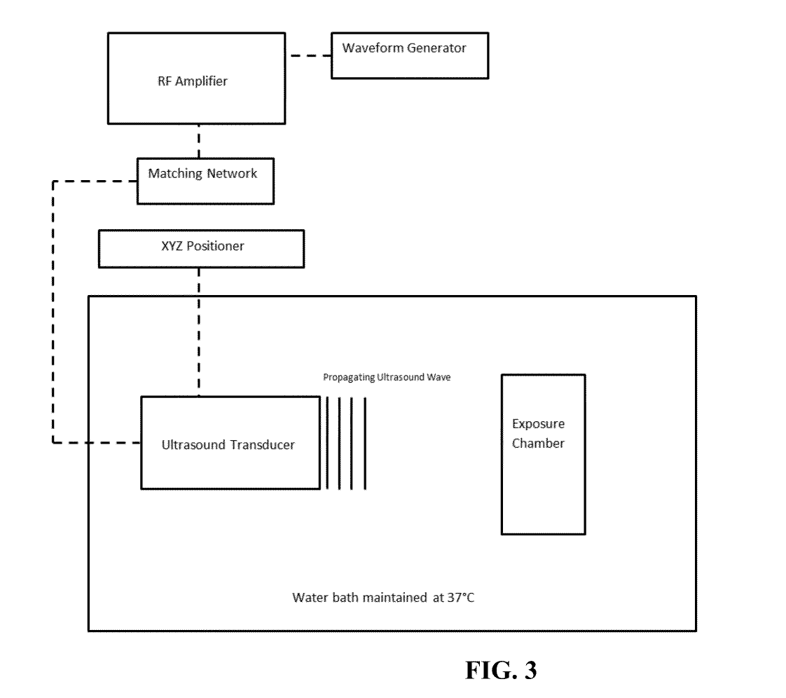

The experimental setup shown in FIG. 2 was modeled in PZFlex modeling software (Weidlinger Associates, Mountain View, Calif., USA) to simulate the range of acoustic pressures to which the cells are exposed to inside the chamber during treatment. FIG. 3 is a schematic of similar components of FIG. 2. The simulation software considers nonlinear wave propagation along with longitudinal and transversal wave propagation. Simulation parameters were established as previously reported (Hensel et al. 2011). Material properties, parameters and dimensions were obtained from our measurements, manufacturers' data and published data. Input properties used in the simulation for polylactic acid (PLA) and Mylar® (materials used to construct the cell exposure chamber) are shown in Table 1.

TABLE 1
Acoustic properties of materials used in simulations
compiled from manufacturer's data and literature.

| | Density | Bulk Modulus | Shear Modulus |
|---|---|---|---|
| Material | (kg/m³) | (MPa) | (MPa) |
| Water (Christensen 1988) | 1000 | 2200 | 0 |
| Polylactic Acid (PLA) | 1251 | 4166.7 | 1286.8 |
| (Jamshidian et al. 2010) | | | |
| Mylar ® (Becker 1965) | 1390 | 7277.8 | 1898.6 |

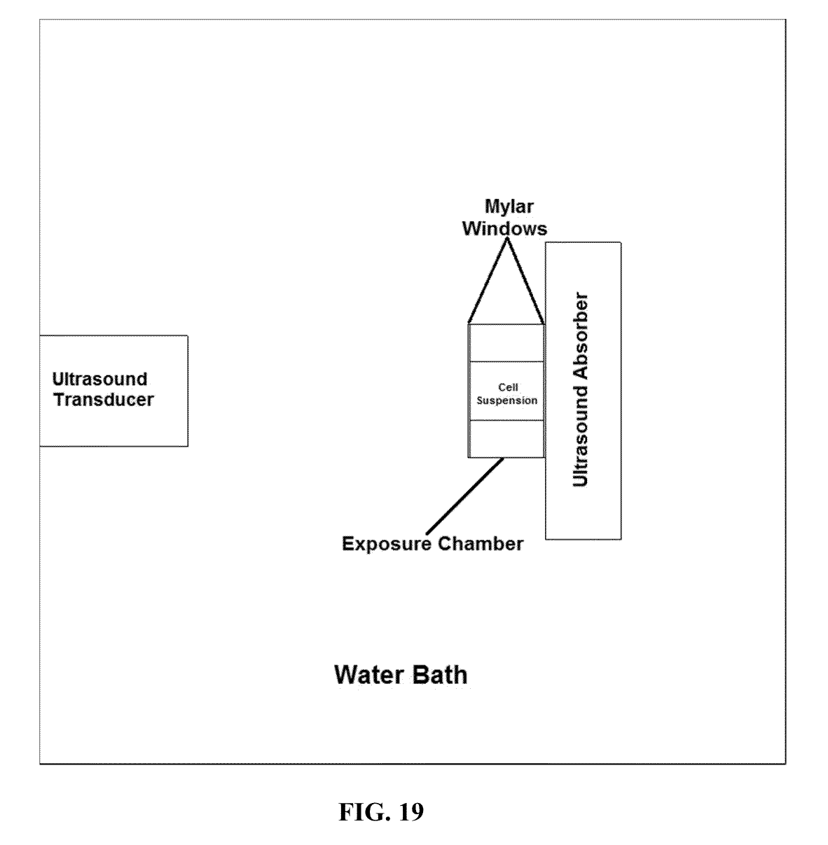

The grid size was set to one fifteenth of the exposure wavelength to ensure proper spatial resolution as recommended by the PZFlex software manufacturer (Nabili et al. 2015). The acoustic absorber was assumed to be a perfect absorber for all frequencies considered. Pressure maps of our experimental setup were generated for the different ultrasound frequencies used experimentally. In order to validate our simulated setup, we compared simulated pressure calculations to experimental measurements obtained with an acoustic hydrophone (HGL-0085, Onda Corporation, Sunnyvale, Calif.). FIG. 19 shows a schematic of the experimental setup as modeled in PZFlex.

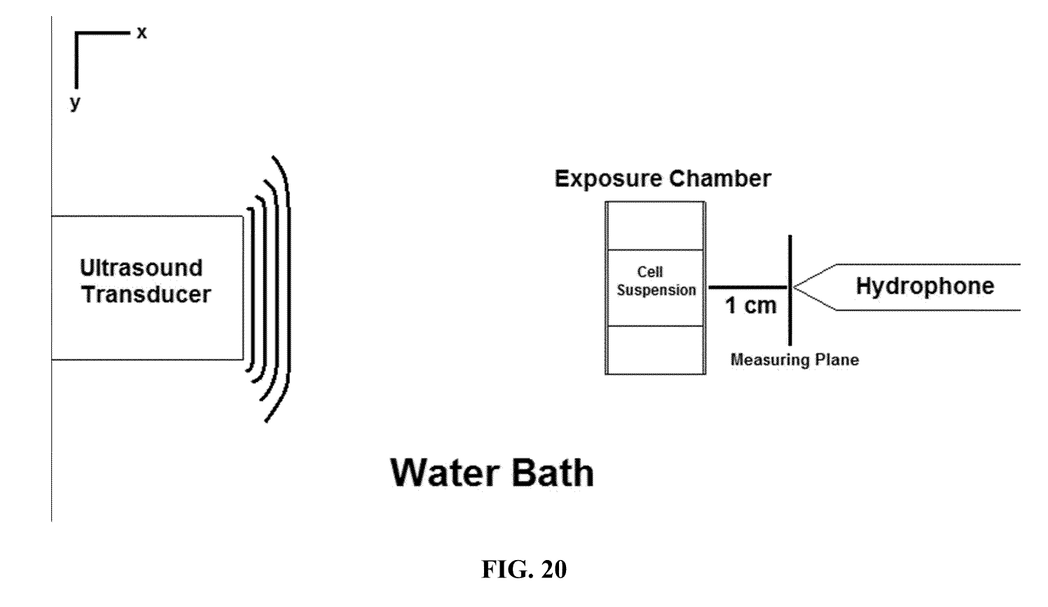

Experimental pressure measurements consisted in placing the acoustic hydrophone at 1 cm behind the exposure chamber and facing the ultrasonic transducer, as shown in FIG. 20.

A 5-cycle ultrasound burst with peak intensity of 1 W/cm² was used for the validation procedure. The hydrophone was swept across the measuring plane in the y-direction in steps of 0.75 mm, as depicted in FIG. 19 and the peak pressure was measured at each location. Similarly, peak pressures across the measuring plane were calculated in our simulations with spatial resolution being one fifteenth of the ultrasound wavelength. Error bars for the simulated data were generated by varying the location of the measuring plane in the x-direction by ±0.2 mm. This procedure was repeated for all frequencies considered in our study.

The cavitation activity inside the chamber was characterized through passive cavitation detection (PCD) for all ultrasound beams used in this study. A single-element transducer (bandwidth of 2.8 MHz to 4.2 MHz; ISO304HP, CTS Valpey Corporation, Hopkinton, Mass.) was aimed at the exposure chamber thus intersecting with the ultrasound beam's path inside the chamber. The signals obtained by the transducer were sent to a spectrum analyzer (MDO3024, Tektronix, Arlington, Va.), and the digitized data was acquired for further analysis in MATLAB. The presence of stable cavitation during ultrasound exposure was determined by identifying the subharmonic and ultraharmonics of all the frequencies used in our study, while the presence of inertial cavitation was determined by identifying the presence of broadband noise across the frequency spectrum of the acquired signal (Leighton 1994). Broadband noise was quantified in two ways. In Method 1, an eighth-order polynomial was fitted to the frequency spectrum in order to omit the signal's harmonic peaks. The fitted signal was then integrated across the detector's bandwidth in a similar manner as described in previous studies (Hong Chen et al. 2014; Rabkin et al. 2005). In Method 2, a frequency window was selectively picked common to all four frequencies used in this study. This window was chosen on the basis of not containing any of the four frequencies' harmonics while being located within the detector's bandwidth (Chen et al. 2003). We picked the window to be from 3.05 MHz to 3.15 MHz and we integrated the region of the spectrum as defined by this window for quantification of inertial cavitation.

Temperature elevations during ultrasound treatments were also monitored by inserting a thermocouple (range: −200° C. to 650° C.; resolution: 0.1° C.; accuracy: 0.1% rdg+0.7° C.) in the exposure chamber during treatment. The signal from the thermocouple was recorded using a dual input thermometer (Wavetek Waterman TMD90). Readings from the thermometer were recorded at t=0 min and every 30 seconds for 10 minutes (n=3).

The number of viable cells was determined using a trypan blue dye exclusion test (Tennant 1964). Ten μL (2-5×106 cells/ml) of each cell sample was acquired and mixed with 10 μL of 0.5% trypan blue solution (Bio-Rad Laboratories, Inc. Hercules, Calif., USA). Ten μL of the mix were acquired and placed on a dual chamber cell counting slide (Bio-Rad Laboratories, Inc. Hercules, Calif., USA). The cell counting slide was then loaded in a TC20 automatic cell counter (Bio-Rad Laboratories, Inc. Hercules, Calif., USA) to determine the proportion of the cells which excluded the dye. Results were presented as the percent ratio of viable cells to the total number of cells in the sample. Ultrasound-treated groups and positive control groups (n=6) were compared to sham groups (n=6) using a two-tail Student t-test with unequal variances.

Cell samples acquired from ultrasound-treated, glucose supplemented and sham groups were centrifuged for 10 min at 1000 rpm and the supernatants were collected for insulin quantification. Insulin concentration in collected supernatants was determined using enzyme-linked immunosorbent assay (ELISA) Insulin Kit (Millipore Corporation, Billerica, Mass., USA) with a SpectraMax M5 Spectrometer (Molecular Devices, Sunnyvale, Calif., USA). Measured insulin concentrations from samples acquired at t=5 min and t=35 min were normalized to their respective initial concentration measured at t=0 min, and data was expressed as fold-change from their initial concentrations. Ultrasound-treated groups were compared to sham groups (n=6) using a two-tail Student t-test with unequal variances. Samples acting as positive controls were suspended in KBS supplemented with 12 mM glucose, a concentration shown to naturally induce insulin secretion in INS-1 cell lines (Hamid, et al. 2002).

In a separate set of experiments, intracellular insulin content in treated and sham groups was determined. Briefly, cell samples acquired from ultrasound-treated and sham groups were washed twice in modified Krebs Ringer bicarbonate buffer (pH 7.4), resuspended in RIPA 1× buffer for lysing and kept on ice for 90 min. Samples were then centrifuged for 10 min at 14000 rpm, and supernatants were collected and stored at −70° C. for subsequent insulin quantification with ELISA. Ultrasound-treated groups were compared to sham groups (n=7) using a two-tail Student t-test with unequal variances.

Results

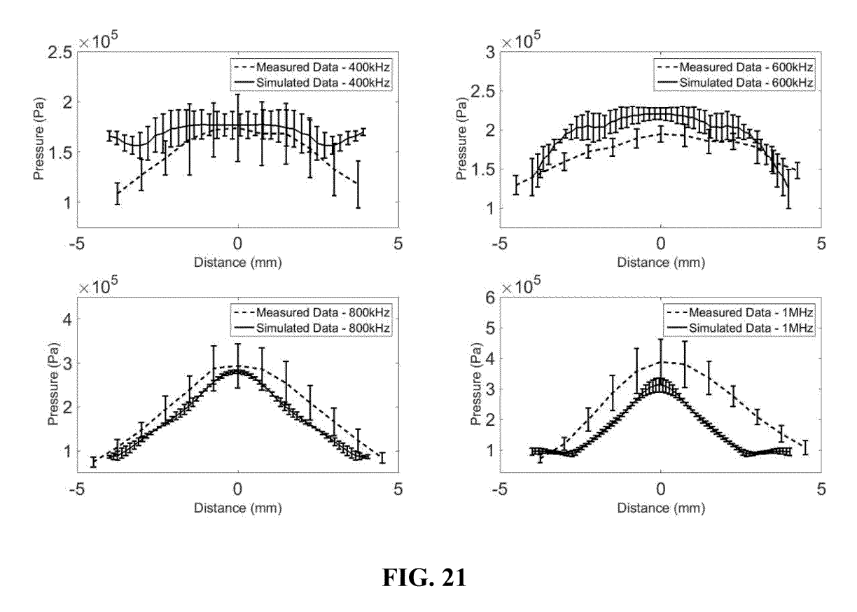

The results of the experimental validation of our simulations are shown in FIG. 21. The x-axis in FIG. 21 corresponds to distances along the measuring plane as depicted in FIG. 20, where 0 mm corresponds to the center of the ultrasound beam. The results show that for all frequencies, the pressure variation as a function of distance follows the same trend in both experimental measurements and simulations.

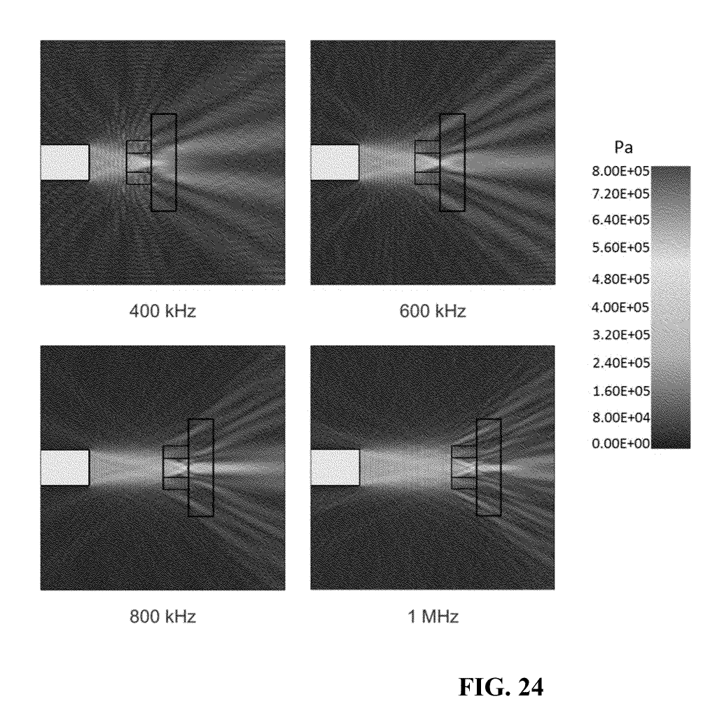

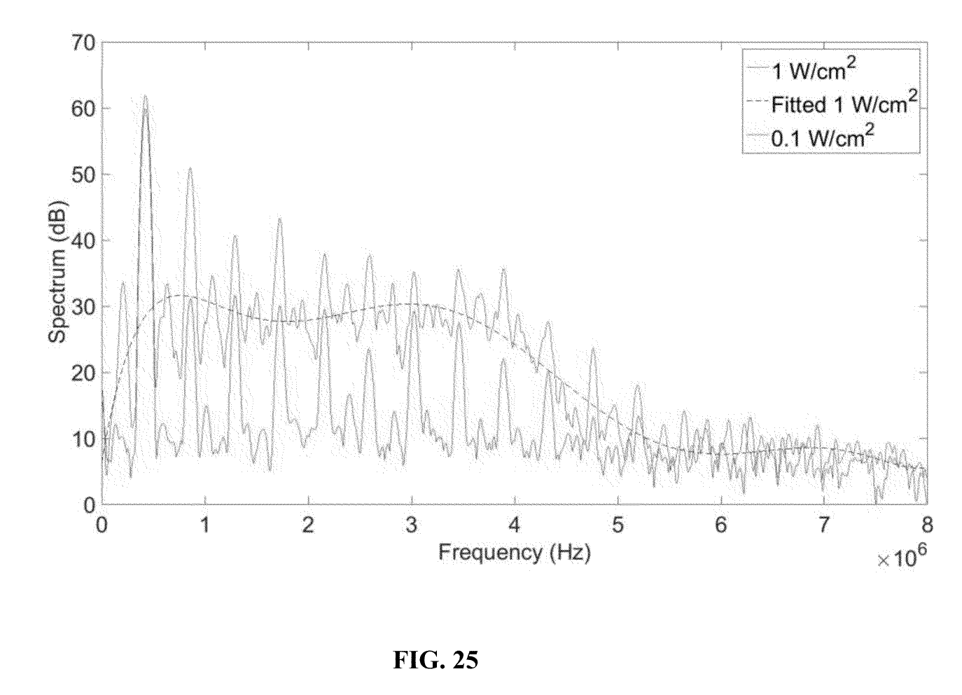

Simulated pressure maps of the experimental setup are displayed in FIG. 24. Simulations showed that cells in the chamber were exposed to mean pressures of 227 kPa±80.23, 218 kPa±90.25, 228 kPa±96.15 and 220 kPa±83.38 when exposed to ultrasound beams with frequencies of 400 kHz, 600 kHz, 800 kHz and 1 MHz, respectively.

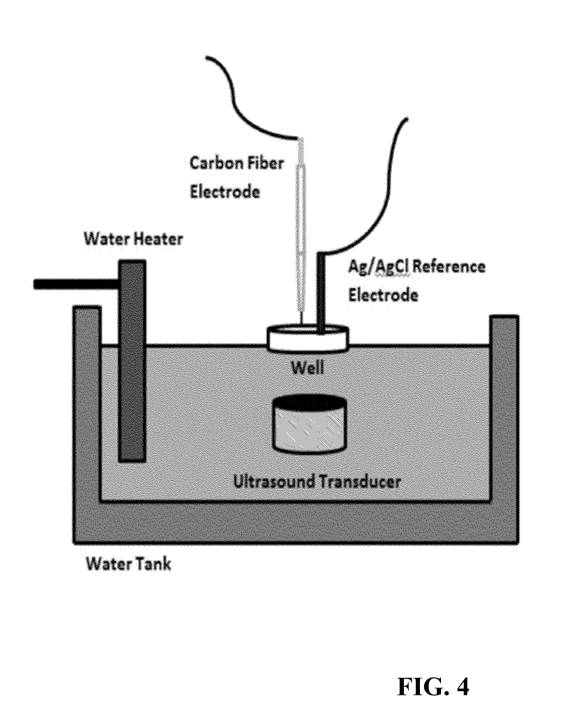

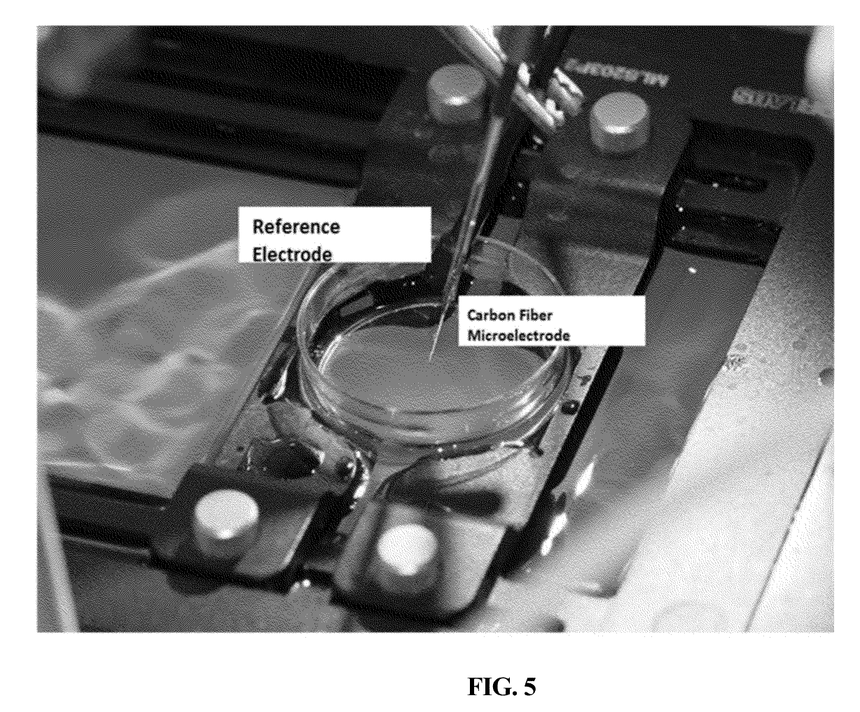

FIG. 4 is a diagram of an experimental setup, according to an embodiment of the invention. FIG. 5 shows an image of a carbon fiber electrode and reference electrode position in the well.

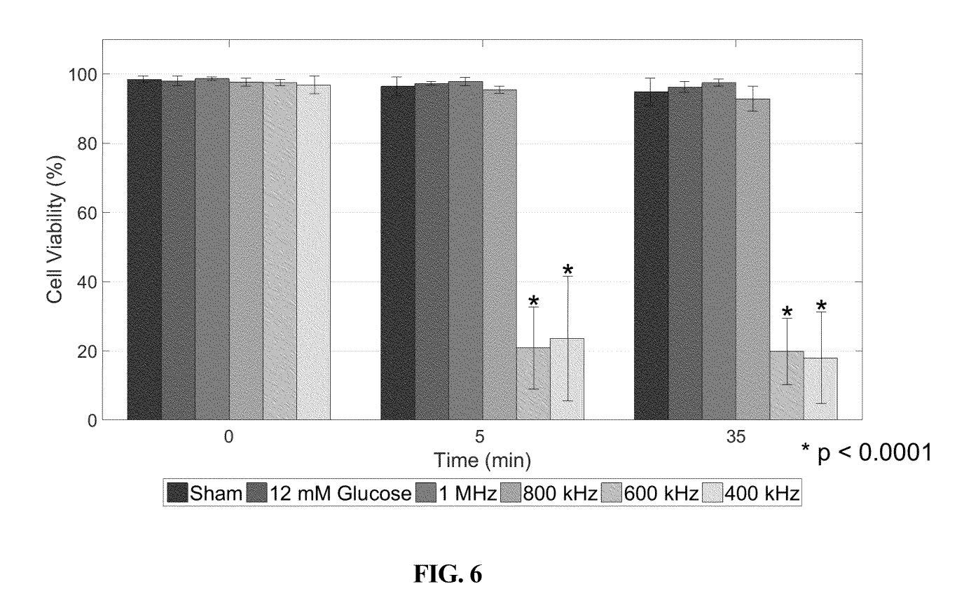

Cell viability studies were performed to assess the safety of the chosen ultrasound parameters (FIG. 6). Results (n=6) of cell viability after ultrasound treatment as measured by trypan blue dye exclusion test. Cell viability was significantly reduced by almost 80% when cells were treated with ultrasound exposures of 400 kHz and 600 kHz respectively (p<0.0001). In contrast, little to no harmful effect was seen in samples treated with 800 kHz and 1 MHz as compared to untreated samples (sham group). In some embodiments, insulin secretagogue glucose had no significant effect on cell viability throughout the experiment.

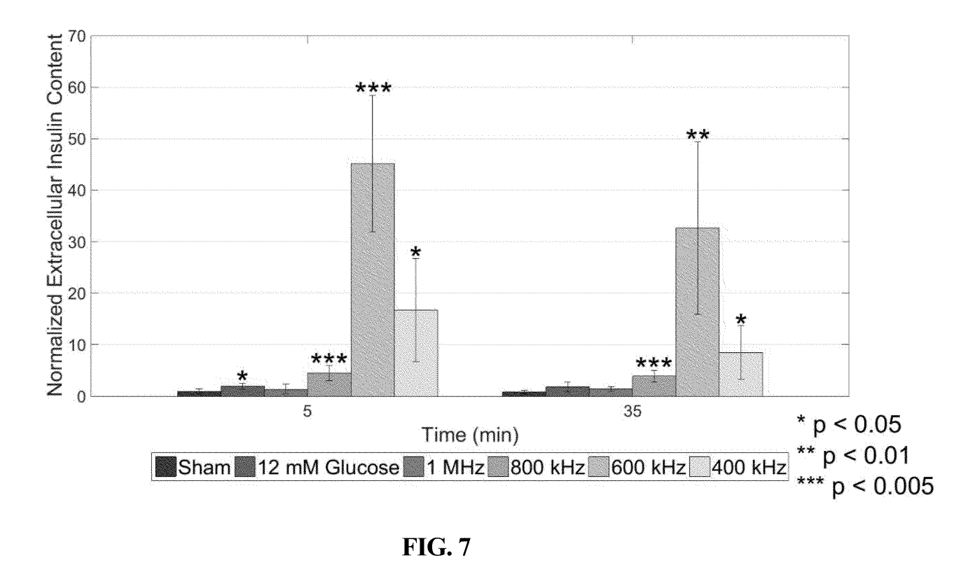

FIG. 7 shows results (n=6) of insulin released into the extracellular space by beta-cells exposed to ultrasound as measured by Insulin ELISA. Measured insulin values at t=5 min and t=35 min were normalized to initial values measured at t=0 min. Changes in extracellular insulin concentration in response to ultrasound treatment are shown in FIG. 7. In some embodiments, insulin levels in the sham group at t=5 min and t=35 min slightly increased (around 25%) from their respective initial concentrations at t=0, representing basal release of insulin from these cells (FIG. 7). However, significant amounts of insulin (>20-fold) were released from beta cells exposed to 400 kHz and 600 kHz ultrasound (p<0.05, FIG. 7) at the cost of cell viability (FIG. 6). At these frequencies, 70% drop in cell viability was observed as compared to sham groups (FIG. 6). Cell exposure to ultrasound frequencies of 800 kHz resulted in a significant 4-fold increase of insulin release (p<0.005, FIG. 6) with no significant effect on cell viability (p>0.005, FIG. 7). In comparison, cells suspended in secretagogue 12 mM glucose (serving as positive controls) showed a 2-fold elevation in extracellular insulin at t=5 and t=35 min, which is consistent with published data (Hamid et al. 2002). These results suggest that ultrasound exposures of 1 W/cm² at a frequency of 800 kHz can safely stimulate insulin secretion from pancreatic beta cells which is within acceptable physiological secretory range for these beta-cells. The cells exposed to 1 MHz ultrasound showed a slight increase in released insulin (around 50%) though no statistical significance was achieved (p>0.05, FIG. 7). However, it is possible that insulin secretion may still be achieved when using 1 MHz ultrasound at higher intensities.

Interestingly, there is a small downward trend in the extracellular insulin content from t=5 min to t=35 min in all of the cases that exhibited enhanced insulin release. This is likely caused by insulin re-uptake by the remaining viable cells which is a common feature in secretory cells, including pancreatic beta-cells. These results demonstrate that ultrasound can be used in a safe and controlled manner.

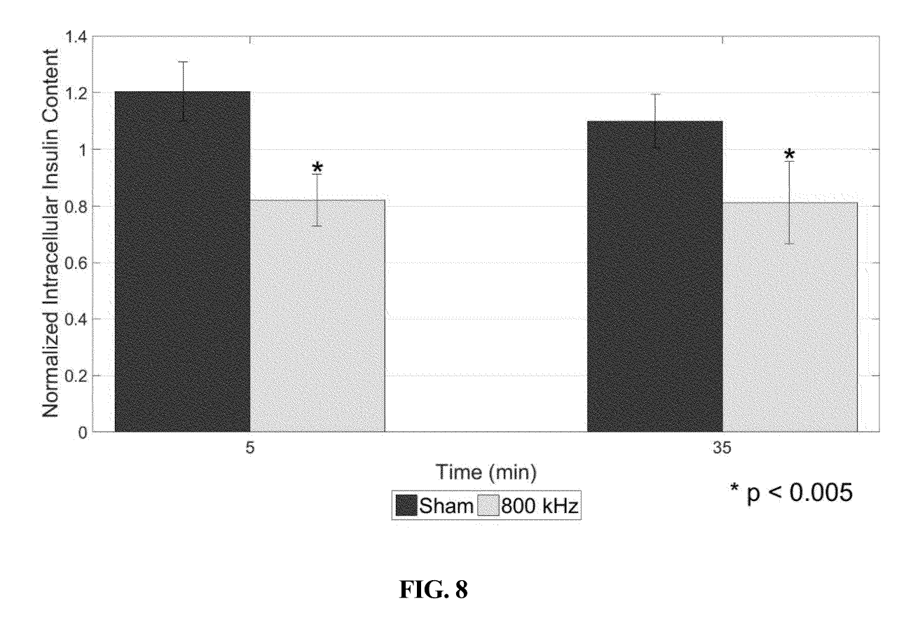

Measurements of intracellular insulin content were consistent with results obtained from the extracellular fluid in our samples (FIG. 8). FIG. 8 shows results (n=6) of intracellular insulin content in beta-cells exposed to ultrasound as measured by Insulin ELISA. Measured insulin values at t=5 min and t=35 min were normalized to initial values measured at t=0 min. Our results showed a reduction of approximately 20% at both t=5 min (p<0.005) and t=35 min (p<0.005) in the insulin content of the cells in ultrasound treated samples (800 kHz) as compared to the sham group, thus indicating increased insulin release from beta cells in response to ultrasound exposure.

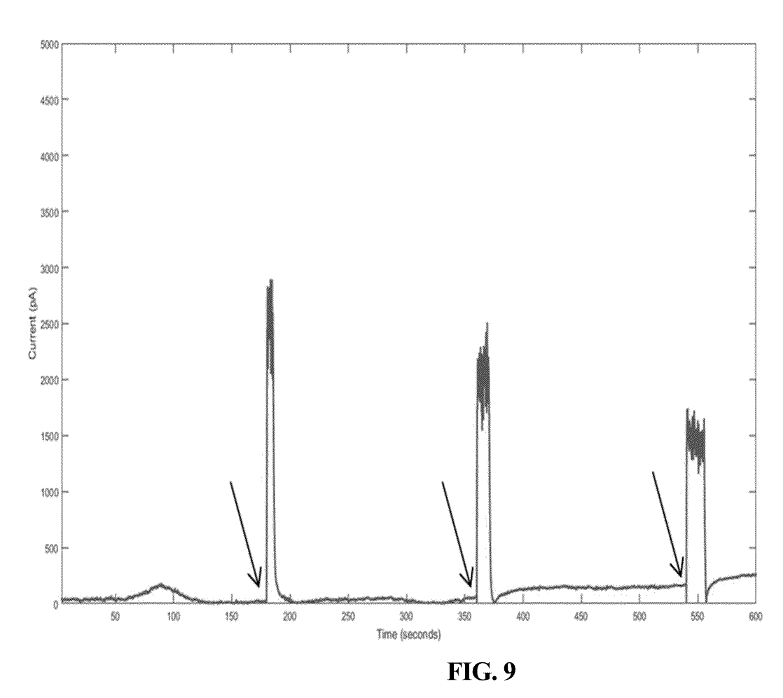

FIG. 9 is an illustration of controlled neurotransmitter release. In FIG. 9, five, ten and fifteen sec long 1 MHz continuous pulses applied at 180, 360 and 540 sec. Amperometric detection of neurotransmitter release mimics the secretion dynamics of insulin in beta cells.

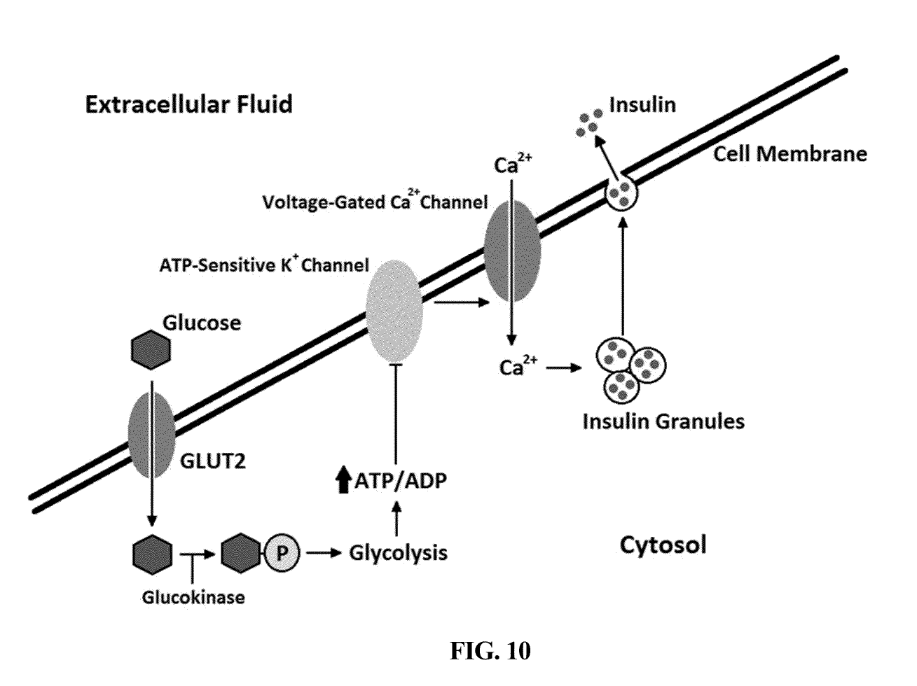

FIG. 10 shows how insulin is naturally secreted from pancreatic β-cells in calcium-dependent manner.

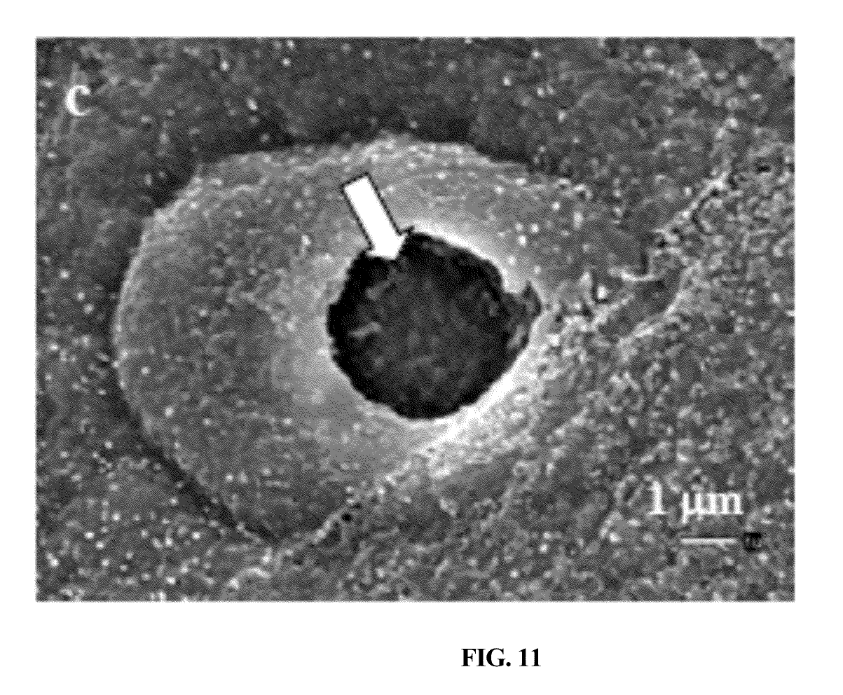

FIG. 11 shows a molecular mechanism schematic of insulin release from beta-cells. SV—secretory vesicle. (courtesy of Dr. Jeremic).

FIG. 12 is an electron micrograph showing a pit formed in an epithelial cell exposed to low-intensity ultrasound (Zderic et al. 2004b).

FIG. 13(a) shows a fluid miscrostreaming around oscillating bubble (Elder 1959).

FIG. 13(b) shows formation of a microjet during inertial cavitation (courtesy of Dr. Lawrence Crum).

FIG. 14 shows that Ionomycin evokes reversible calcium transients in cultured rat pancreatic beta cells.

FIG. 15 shows an experimental setup for ultrasound stimulation of pancreatic beta cells.

FIG. 16 shows passive cavitation detection showed presence of indicators of stable cavitation (arrows) and inertial cavitation (broadband noise) at 0.9 MHz and 0.5 W/cm² (Zderic et al. 2004a).

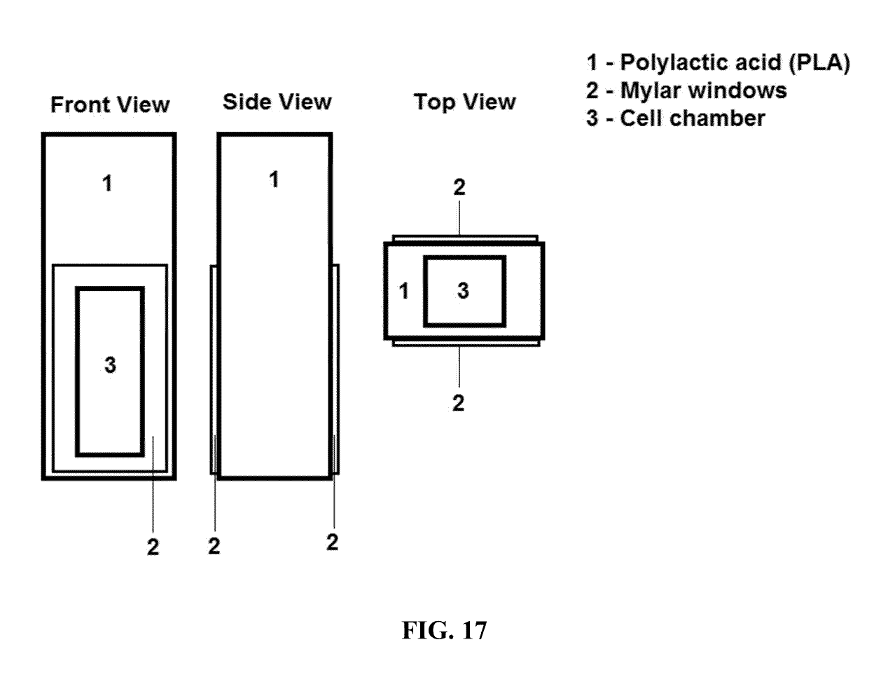

FIGS. 17 and 18 shows a 3D printed exposure chamber with Mylar windows.

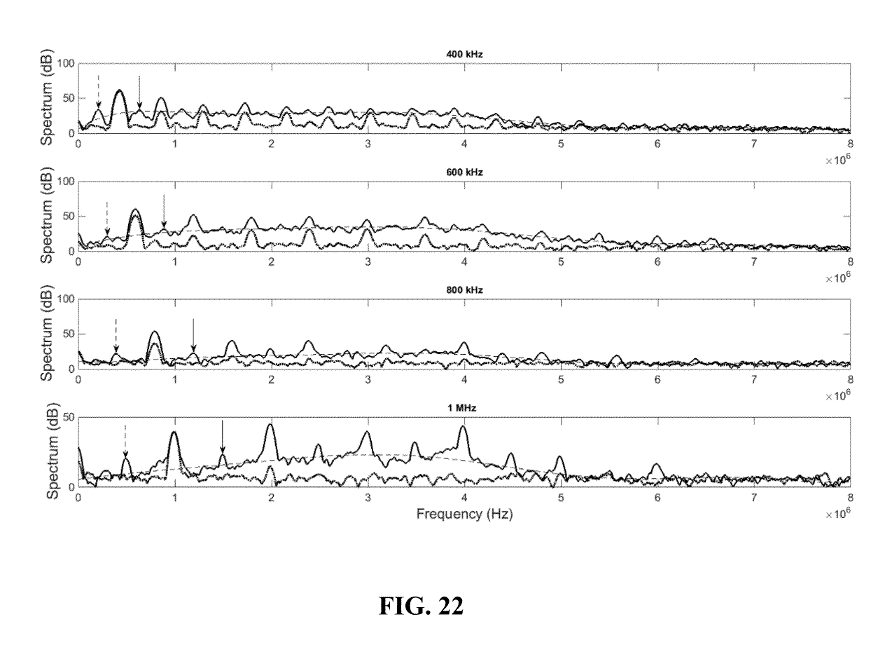

Results of the acoustic cavitation study are shown in FIG. 22. Stable cavitation, as measured by the presence of the frequencies' subharmonics and ultraharmonics (dashed and solid black arrows respectively), was shown to be present inside the chamber for all four ultrasound frequencies used. We also observed inertial cavitation in all four spectra as shown by the presence of broadband noise and estimated by the eighth-order polynomial fitting of the four frequency spectra (dashed black line). The fitting of all four spectra was calculated to have R2 of over 0.95. To highlight the presence of broadband noise, we generated spectra for all frequencies at an intensity of 0.1 W/cm² (gray solid line), which can generate little to no inertial cavitation.

The results of inertial cavitation measurements are shown in Table 2.

TABLE 2
Quantification of inertial cavitation for 400 kHz,
600 kHz, 800 kHz and 1 MHz from measured spectra.

| Frequency | Method 1 (dB) | Method 2 (dB) |
|---|---|---|
| 400 kHz | 1.55 × 10⁸ | 2.85 × 10⁶ |
| 600 kHz | 1.73 × 10⁸ | 2.99 × 10⁶ |
| 800 kHz | 1.13 × 10⁸ | 2.19 × 10⁶ |
| 1 MHz | 1.02 × 10⁸ | 2.12 × 10⁶ |

It can be seen in both methods 1 and 2, that there is a distinct increase in measured inertial cavitation in the spectra corresponding to 400 kHz and 600 kHz ultrasound when compared to the spectra of higher frequencies, suggesting that this increase in inertial cavitation may be involved in the significant reduction in the cell viability observed in FIG. 6.

FIG. 22 shows spectra obtained from 400 kHz, 600 kHz, 800 kHz and 1 MHz ultrasound for passive cavitation detection. Subharmonics (dashed black arrows), ultraharmonics (solid black arrows) and broadband noise (dashed black curve) were observed in all spectra obtained at an intensity of 1 W/cm2 (solid black curve). Spectra obtained at an intensity of 0.1 W/cm2 is represented by the solid gray curve.

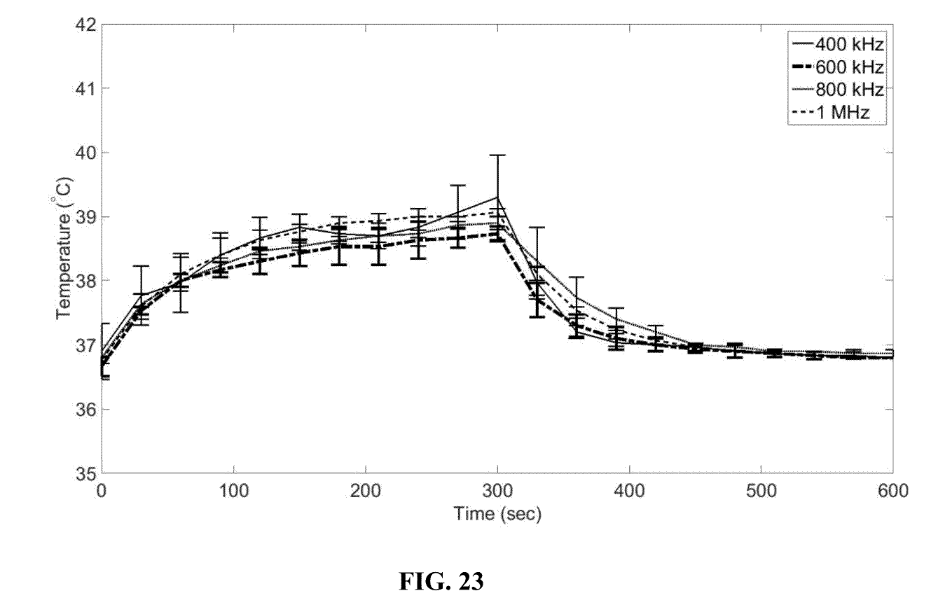

FIG. 23 shows measurements of temperature elevations in the exposure chamber during ultrasound treatment for all four frequencies under study. It can be seen that a 5 minute ultrasound exposure caused an elevation no higher than 3° C. on average for all four frequencies. Furthermore, the trend appears to be the same in all cases thus potentially indicating that the observed differences in cell viability and insulin release among different ultrasound frequencies is due to mechanical rather than thermal effects. Thus, FIG. 23 shows temperature measurements inside the cell chamber during ultrasound treatment for frequencies of 400 kHz, 600 kHz, 800 kHz and 1 MHz (n=3).

FIG. 24 shows modeling of acoustic pressure maps using PZFlex. In FIG. 24, simulated pressure maps of the experimental setup for frequencies of 400 kHz, 600 kHz and 1 MHz (view from top).

### DISCUSSION

The results of our experiments suggest that ultrasound exposure can stimulate insulin release from pancreatic beta-cells in a safe and controlled manner. Our experiments showed that ultrasound applied at an intensity of 1 W/cm² and frequencies of 1 MHz and 800 kHz appears to have no significant effect on cell viability. However, 800 kHz ultrasound showed a significant (4-fold) increase in insulin release from the beta-cells, whereas the cells exposed to 1 MHz ultrasound showed a lesser (50% on average) increase in insulin release, both of which could be useful in fighting hyperglycemia in diabetics. It is possible that 1 MHz ultrasound can also safely stimulate significant insulin release from beta-cells at a higher intensity.

Our data also show that insulin release stops immediately after ultrasound treatment, thus highlighting the fact that ultrasound-induced insulin release can be controlled. It is important to note here that 1 MHz and 800 kHz ultrasound stimulation produced a comparable secretory stimulatory response in beta-cells evoked by natural secretagogue glucose. This is important because too much insulin release can also be harmful to diabetics as it can lead hypoglycemia. Cells exposed to lower frequencies of 400 kHz and 600 kHz experienced significant loss in cell viability (approximately 80%) which resulted in significant amounts of insulin released into the extracellular space. Thermal measurements showed that 5 minute ultrasound exposure raised the temperature of the cell medium by no higher than 3° C. for all frequencies considered, which is unlikely to have any damaging effect on the cells. Our study of cavitation activity showed that stable cavitation and inertial cavitation were present in the cell chamber when ultrasound was applied at all frequencies under study. However, lower frequencies of 400 kHz and 600 kHz exhibited distinctively higher levels of inertial cavitation compared to 800 kHz and 1 MHz frequencies, suggesting that higher levels of inertial cavitation may play an increasingly detrimental role in cell viability. In our current studies we did not correlate cavitation activity to enhanced insulin release observed in beta-cells exposed to 800 kHz ultrasound and therefore further studies are required to determine the exact mechanisms involved in ultrasound-enhanced insulin release from pancreatic beta-cells. Nonetheless, in addition to cavitation, other mechanisms documented in literature could play an important role in this process.

Insulin is naturally secreted from pancreatic beta-cells in calcium-dependent manner. As mentioned previously, calcium influx is the last triggering step before exocytosis of insulin containing vesicles in glucose-stimulated insulin secretion (GSIS). Furthermore, ultrasound induced-bioeffects have been shown to produce intracellular calcium transients in various cell types which have triggered Ca2+-dependent exocytosis of secretory vesicles. Tyler et al. (2008) showed in an ex vivo study that low-intensity, low-frequency ultrasound was capable of activating Ca2+ transients followed by SNARE-mediated synaptic vesicle exocytosis. Another study showed that ultrasound exposure of chromaffin cells was capable of releasing catcholamines via Ca2+-mediated exocytosis (Robinson et al. 1996). It is therefore possible that the observed increase in insulin secretion from pancreatic beta-cells when treated with ultrasound is the result of ultrasound-induced calcium transients and subsequent triggering of insulin vesicle exocytosis. Calcium currents have also been shown to be essential to a cell's resealing process after exposure to acoustic cavitation and other mechanical stresses generated by 24 kHz ultrasound exposures (Schlicher et al. 2006).

Ultrasound-induced changes in the cell membrane permeability have been strongly correlated with cavitation activity, which leads to formation of reversible pits in the cell membranes thus allowing delivery of genes, drugs, and macromolecules into the cells or release of the cell components (Guzmán et al. 2002; Zderic et al. 2004a; Zderic et al. 2004b). Specifically, a study showed that cavitation generated by ultrasound enhanced cellular incorporation of macromolecules up to 28 nm in radius through repairable micron-scale holes in the membrane of DU 145 prostate-cancer cells which were shown to reseal after 1 minute (using native cell healing response involving endogenous vesicle-based membrane resealing)(Schlicher et al. 2006). Tsukamoto et al. (2011) demonstrated that cytoplasmic calcium in fibroblasts cultured in vitro was increased in response to stable cavitation generated by exposure to 1 MHz pulsed ultrasound. In a similar study, Mortimer et al. (1988) showed that ultrasound treatment increased calcium uptake in 3T3 fibroblasts by almost 20% after a 5 minute exposure. Similar ultrasound effects could potentially lead to the formation of reversible pits in the membrane of beta-cells, creating a calcium influx and the subsequent release of insulin. Our study on cavitation activity showed the presence of both stable and inertial cavitation in ultrasound at frequencies that showed increased insulin release from pancreatic beta-cells. It is therefore possible that either one or both types of cavitation activity are involved in ultrasound enhanced-insulin release from pancreatic beta-cells though further studies are required to confirm it.

Other possible mechanisms responsible for enhanced membrane permeability and subsequent insulin release include mechanical stimulation of mechano-sensitive proteins in the plasma membrane of the beta-cell. Studies have shown that ionic mechanisms other than the inhibition of KATP channels may be involved in membrane depolarization caused by higher glucose concentrations. One of these mechanisms is believed to be beta-cell swelling induced by high concentration glucose and dependent on glucose metabolism (Helen et al. 2007; Semino et al. 1990; Takii et al. 2006). beta-cell swelling is believed to be caused by increased intracellular lactate (Best 1999), Na+ and Cl— (Best et al. 1997) concentrations due to increased beta-cell metabolic activity, leading to intracellular hyperosmolarity and ultimately, insulin secretion. Increased insulin secretion caused by osmotic cell swelling has been attributed to stimulation of stretch-activated cation channels (SAC) sensitive to mechanical stretching of the plasma membrane (Best et al. 2010) and volume-regulated anion channels (VRAC) sensitive to cell volume changes due to hypotonicity-induced cell swelling (Takii et al. 2006). Therefore, it is possible that SAC and VRAC channels are activated by physical and subcellular perturbations of the beta-cell structure in response to ultrasound exposure. Activation of these channels could in turn be responsible for membrane depolarization, activation of voltage-gated Ca2+ channels and subsequent insulin secretion.

Many studies have been aimed at identifying ultrasound-mediated bioeffects that can mechanically activate membrane proteins and modulate intracellular pathways, a process often referred to as mechanotransduction. In particular, low-intensity ultrasound was shown to cause morphological changes to neuronal cells, a process that the authors believe could have implications in neuronal cell growth and other downstream cellular processes mediated by the cytoskeleton of the cell (Hu et al. 2013; Hu et al. 2014). Cells were shown to recover their original pre-exposure size within 30 minutes after the end of exposure. Transient changes in cell morphology and cytoskeletal disruptions caused by ultrasound exposure could stimulate machano-sensitive membrane proteins (VRAC or SAC), depolarizing the membrane to levels necessary to open Ca2+ channels and consequently stimulate insulin secretion. Another effect of ultrasound that could play a role in modulating membrane channels is a process known as “intramembrane cavitation”. Krasovitski et al. (2011) suggested that the cell membrane is capable of transforming oscillating acoustic pressure waves into intracellular deformations. Such cyclic deformations could stimulate cycles of stretch and release in the cell membrane and the cytoskeleton, which could in turn stimulate mechano-sensitive proteins, increase membrane permeability and depolarize the cell's membrane. Other ultrasound bioeffects that could play important roles in beta-cell stimulation include cell responses to mechanical stresses caused by acoustic radiation force (Morris and Juranka 2007).

Finally, it is also possible that enhanced insulin release resulting from ultrasound exposure is the result of insulin granules leaking out of the cell through transient membrane pores created by ultrasound cavitation. It is estimated that around 700 out of the total 10000 insulin containing granules are docked to the plasma membrane, 200 of which are primed and readily releasable (Olofsson et al. 2002). Granules that are docked and primed at the beta-cell's membrane are said to belong to the readily releasable pool (RRP), while the rest are considered to belong to the reserve pool (RP) (Olofsson et al. 2002). Therefore, it is possible that transient poration of the plasma membrane caused by acoustic cavitation may also be permeating the cell membrane to either insulin leaking directly from the RRP, or insulin granules being released into the extracellular space and subsequently being destroyed by ultrasound bioeffects. Time-dependent studies of insulin release dynamics will resolve this issue.

Cell viability as assessed by trypan blue dye exclusion test provides a measure of cell viability by assessing plasma membrane integrity. This measurement is of great relevance to our study since we hypothesize that transient disruption of the plasma membrane may play a role in ultrasound-enhanced insulin secretion. However, in vitro studies have shown that certain ultrasound exposures can result in other bioeffects that can be harmful to the cells. In particular, it has been observed that cells can become apoptotic or necrotic through Ca2+-dependent pathways and/or mitochondria-caspase pathways when exposed to ultrasound exposures inducing inertial cavitation (Honda et al. 2004; Kumon et al. 2009). However, careful optimization of frequencies and duration of stimulations can be effectively used to stimulate insulin-release by non-invasive methods while retaining cell viability as our results show.

In FIG. 6, Results (n=6) of cell viability after ultrasound treatment as measured by trypan blue dye exclusion test.

Conclusion: If shown successful our approach may eventually lead to new methods in the treatment of diabetes and other secretory diseases. Our future studies will focus on application of ultrasound to the pancreas in an in vivo animal model to determine whether it would be possible to stimulate beta cells without stimulating other endocrine and exocrine cells of the pancreas.

Low-intensity ultrasound energy can be used to safely stimulate insulin release from pancreatic beta-cells in an in vitro environment. Our findings show that ultrasound, at least when applied at a frequency of 800 kHz and intensity of 1 W/cm², can induce insulin secretion from beta-cells similar to secretagogue glucose while preserving cell viability. The mechanisms by which ultrasound can lead to enhanced insulin secretion will be studied in future experiments. Experiments aimed at fully understanding the ultrasound-induced bioeffects involved in this process and their role in modulating Ca2+ dynamics are needed and will follow.

What is claimed is:

1. A pancreatic beta cell stimulation system for stimulating release of insulin from pancreatic beta cells, comprising:
an ultrasonic transducer configured to be acoustically coupled to a body of a user; and
an ultrasound controller configured to be in communication with said ultrasonic transducer so as to provide control signals to said ultrasonic transducer during operation,
wherein said ultrasound controller is further configured to generate said control signals based on a planned amount of stimulation of pancreatic beta cells within said body of said user such that said control signals instruct said ultrasonic transducer to transmit ultrasound waves having selected intensity and frequency calculated to cause stimulation of said pancreatic beta cells.

2. The pancreatic beta cell stimulation system of claim 1, further comprising a data storage device configured to be in communication with said ultrasound controller during operation,
wherein said data storage device contains information concerning ultrasound stimulation of pancreatic beta cells,
wherein said ultrasound controller receives and uses said information concerning ultrasound stimulation from said data storage device to generate said control signals.

3. The pancreatic beta cell stimulation system of claim 2, wherein said ultrasound controller is implemented on a mobile device that is in wireless communication with said ultrasonic transducer.

4. The pancreatic beta cell stimulation system of claim 1, further comprising a glucose sensing device that is configured to be in communication with said ultrasound controller, said glucose sensing device being attachable to said body of said user so as to sense information concerning glucose in a blood stream of said user and to provide a glucose information signal to said ultrasound controller,
wherein said ultrasound controller receives and uses said glucose information signal to generate said control signals.

5. The pancreatic beta cell stimulation system of claim 4, wherein at least one of said ultrasonic transducer and said glucose sensing device is at least partially implantable within said body of said user.

6. The pancreatic beta cell stimulation system of claim 1, wherein said ultrasound controller is further configured to generate said control signals based on said planned amount of stimulation of pancreatic beta cells within said body of said user such that said control signals instruct said ultrasonic transducer to transmit ultrasound waves for a selected duration of said stimulation of said pancreatic beta cells.

7. The pancreatic beta cell stimulation system of claim 1, wherein said selected frequency is in a range of about 100 kHz to 5.0 MHz.

8. The pancreatic beta cell stimulation system of claim 1, wherein said selected frequency is in a range of about 400 kHz to 1.0 MHz.

9. The pancreatic beta cell stimulation system of claim 1, wherein said selected frequency is about 800 kHz.

10. The pancreatic beta cell stimulation system of claim 1, wherein said ultrasonic transducer is structured to be at least partially focusing to provide at least a degree of focusing onto a sub-volume of said body of said user where pancreatic beta cells are expected to be present.

11. The pancreatic beta cell stimulation system of claim 10, wherein said ultrasonic transducer is an array of transducer elements configured to be electronically focusable and electronically steerable.

12. The pancreatic beta cell stimulation system of claim 10, wherein said sub-volume of said body of said user contains at least a portion of said user's pancreas.

13. The pancreatic beta cell stimulation system of claim 10, wherein said sub-volume of said body of said user contains at least a portion of implanted pancreatic beta cells.

14. A method of stimulating insulin release from pancreatic beta cells within a body of a subject, comprising:
determining intensity and frequency for exposure of pancreatic beta cells within said body of said subject based on a planned stimulation of pancreatic beta cells within said body of said user; and
exposing said pancreatic beta cells within said body of said subject to ultrasound waves using the determined intensity and frequency.

15. The method of claim 14, further comprising storing information concerning ultrasound stimulation of said pancreatic beta cells,
wherein said determining is based on said information concerning ultrasound stimulation from said data storage device.

16. The method of claim 14, wherein said exposing said pancreatic beta cells is implemented by an ultrasonic transducer that is configured to be acoustically coupled to said body of said user.

17. The method of claim 16, further comprising sensing glucose information from a blood stream of said user using a glucose sensing device,
wherein said glucose information is used in said determining.

18. The method of claim 17, further comprising implanting at least a portion of at least one of said glucose sensing device and ultrasonic transducer into said body of said user.

19. The method of claim 16, further comprising:
generating control signals based on a planned amount of stimulation of pancreatic beta cells within said body of said user such that said control signals instruct said ultrasonic transducer to transmit ultrasound waves for a selected duration of said stimulation of said pancreatic beta cells.

20. The method of claim 14, wherein said selected frequency is in a range of about 100 kHz to 5.0 MHz.

21. The method of claim 14, wherein said selected frequency is in a range of about 400 kHz to 1.0 MHz.

22. The method of claim 14, wherein said selected frequency is about 800 kHz.

23. The method of claim 14, further comprising at least partially focusing at least a degree of focusing onto a sub-volume of said body of said user where pancreatic beta cells are expected to be present.

24. The method of claim 23, wherein said sub-volume of said body of said user contains at least a portion of said user's pancreas.

25. The method of claim 23, wherein said sub-volume of said body of said user contains at least a portion of implanted pancreatic beta cells.
